(19) United States
(12) Patent Application Publication
(10) Pub. No.: US 2026/0137497 A1
(43) Pub. Date: May 21, 2026

(54) Tooth Whitening System, Kit, And/Or Method

(71) Applicant: Colgate-Palmolive Company, New York, NY (US)

(72) Inventor: Leighton DAVIES-SMITH, Lebanon, NJ (US)

(73) Assignee: Colgate-Palmolive Company, New York, NY (US)

(21) Appl. No.: 19/389,597

(22) Filed: Nov. 14, 2025

Related U.S. Application Data
(60) Provisional application No. 63/721,743, filed on Nov. 18, 2024.

(51) Int. Cl. A61C 19/06; A61N 5/06

(52) U.S. Cl. CPC A61C 19/066; A61N 5/0603; A61N 5/062; A61N 2005/0606; A61N 2005/0643; A61N 2005/0663

(57) ABSTRACT

A tooth whitening system, kit, and/or method. The tooth whitening system may include a whitening device and a mouth tray that are detachably coupled together. The whitening device may include a mouthpiece with one or more first connection features and a light emitting assembly that is configured to generate light and emit the light from the front surface of the mouthpiece. The mouth tray may include a support wall having a front surface and a rear surface, a bite platform extending from the front surface, and one or more second connection features on the rear surface. The mouthpiece of the whitening device may be detachably coupled to the mouth tray by engagement between the one or more connection features of the mouthpiece and the one or more second connection features of the mouth tray.

### CROSS-REFERENCED TO RELATED APPLICATIONS

The present application claims the benefit of priority to U.S. Provisional Patent Application Ser. No. 63/721,743, filed Nov. 18, 2024, the entirety of which is incorporated herein by reference.

### BACKGROUND

Tooth whitening devices and systems have been in use for many years. Some such devices and systems use a combination of a whitening composition applied onto the user's teeth followed by emitting a light onto the user's teeth that are pre-coated with the whitening composition. Tooth whitening may be done under the supervision of a dental practitioner, or at home using a retail product. A need exists for tooth whitening systems, kits, and/or methods that provide flexibility in their use.

### BRIEF SUMMARY

The present invention is directed to a tooth whitening system, a tooth whitening kit, and/or a tooth whitening method. The tooth whitening system may include a whitening device and a mouth tray that are detachably coupled together. The whitening device may include a mouthpiece with one or more first connection features and a light emitting assembly that is configured to generate light and emit the light from the front surface of the mouthpiece. The mouth tray may include a support wall having a front surface and a rear surface, a bite platform extending from the front surface, and one or more second connection features. The mouthpiece of the whitening device may be detachably coupled to the mouth tray by engagement between the one or more connection features of the mouthpiece and the one or more second connection features of the mouth tray.

In one aspect, the invention may be a tooth whitening system comprising: a whitening device comprising: a mouthpiece comprising a front surface and one or more first connection features; and a light emitting assembly comprising one or more light emitters that are configured to generate light which is emitted from the front surface of the mouthpiece; a mouth tray comprising: a support wall comprising a front surface and a rear surface; a bite platform extending from the front surface of the support wall; and one or more second connection features; wherein the mouthpiece of the whitening device is configured to be detachably coupled to the mouth tray by engagement between the one or more first connection features of the mouthpiece and the second connection feature of the mouth tray.

In another aspect, the invention may be a tooth whitening kit comprising: a whitening device comprising: a mouthpiece comprising a front surface; a light emitting assembly comprising one or more light emitters that are configured to generate light which is emitted from the front surface of the mouthpiece; and one or more first connection features; a mouth tray comprising one or more second connection features, the mouth tray comprising an upper channel configured to receive a user's upper teeth during use and a lower channel configured to receive the user's lower teeth during use, wherein the whitening device and the mouth tray are configured to be detachably coupled together via engagement between the one or more first connection features of the whitening device and the one or more second connection features of the mouth tray; and at least one dispensing unit containing a tooth whitening composition.

In yet another aspect, the invention may be a tooth whitening method comprising: detachably coupling a whitening device to a mouth tray that is distinct from the whitening device, the whitening device comprising a mouthpiece comprising a front surface and at least one light emitter, the mouth tray comprising a support wall and a bite platform that define an upper channel and a lower channel; introducing a tooth whitening composition into the upper and lower channels of the mouth tray; inserting the mouth tray into a user's mouth so that upper teeth of the user are located within the upper channel of the mouth tray and lower teeth of the user are located within the lower channel of the mouth tray; and activating the whitening device so that the at least one light emitter generates light and emits the light from the front surface of the mouthpiece and through the support wall of the mouth tray and onto the upper and lower teeth of the user.

In still another aspect, the invention may be a tooth whitening system comprising: a whitening device comprising: a mouthpiece comprising a front surface; and a light emitting assembly comprising one or more light emitters that are configured to generate light which is emitted from the front surface of the mouthpiece; a mouth tray comprising: a support wall comprising a front surface and a rear surface; and a bite platform extending from the front surface of the support wall; wherein the mouthpiece of the whitening device is configured to be detachably coupled to the mouth tray by engagement between the mouthpiece of the whitening device and the support wall of the mouth tray.

In a further aspect, the invention may be a tooth whitening system comprising: a whitening device comprising: a mouthpiece comprising a front surface; and a light emitting assembly comprising one or more light emitters that are configured to generate light which is emitted from the front surface of the mouthpiece; a mouth tray comprising: a support wall comprising a front surface and a rear surface; and a bite platform extending from the front surface of the support wall, the bite platform and the front surface of the support wall defining an upper channel and a lower channel; wherein in a first use configuration, the mouthpiece of the whitening device is positioned within a user's mouth and the one or more light emitters are powered to emit the light onto the user's teeth; and wherein in a second use configuration, the whitening device is coupled to the mouth tray, the mouth tray is positioned within the user's mouth so that upper teeth of the user are located within the upper channel of the mouth tray and lower teeth of the user are located within he lower channel of the mouth tray, and the one or more light emitters are powered to emit the light onto the user's teeth.

Further areas of applicability of the present invention will become apparent from the detailed description provided hereinafter. It should be understood that the detailed description and specific examples, while indicating the preferred embodiment of the invention, are intended for purposes of illustration only and are not intended to limit the scope of the invention.

### BRIEF DESCRIPTION OF THE DRAWINGS

The present invention will become more fully understood from the detailed description and the accompanying drawings, wherein:

FIG. 1 is a schematic view of a kit containing a whitening device, a mouth tray, a sheet for a custom mouth tray, and dispensing units containing a tooth whitening composition;

FIG. 2 is a front perspective view of the whitening device of FIG. 1;

FIG. 3 is an exploded front perspective view of the whitening device of FIG. 1;

FIG. 4 is a cross-sectional view taken along line IV-IV in FIG. 2;

FIG. 5 is a view illustrating the whitening device operably coupled to an external electronic device to power the whitening device;

FIG. 6 is a view illustrating the whitening device placed inside of a user's mouth;

FIG. 7 is a rear perspective view of the mouth tray of FIG. 1;

FIG. 8 is a front view of the mouth tray of FIG. 2;

FIG. 9A is a perspective view illustrating the whitening device and the mouth tray with arrows showing how to bend or fold the whitening device to facilitate attachment to the mouth tray in accordance with an embodiment of the invention;

FIG. 9B is a perspective view illustrating the whitening device and the mouth tray with the whitening device folded along the arrows from FIG. 9B;

FIG. 9C is a perspective view illustrating the whitening device of FIG. 2 being detachably coupled to the mouth tray of FIG. 7;

FIG. 9D is a perspective view illustrating the whitening device of FIG. 2 and the mouth tray of FIG. 7 in an attached state;

FIG. 10 illustrates the whitening device and the mouth tray in an attached state and positioned within a user's mouth for a treatment;

FIG. 11 is a front perspective view of a whitening device in accordance with an alternative embodiment;

FIG. 12 is a front perspective view of a mouth tray in accordance with an alternative embodiment;

FIG. 13A illustrates the whitening device of FIG. 11 in preparation for being detachably coupled to the mouth tray of FIG. 12;

FIG. 13B illustrates the whitening device of FIG. 11 and the mouth tray of FIG. 12 in a detachably coupled state;

FIG. 14 illustrates a tooth whitening system that includes a whitening device and a mouth tray in an attached configuration in accordance with another embodiment of the present invention;

FIG. 15 illustrates the tooth whitening system of FIG. 15 with the whitening device and the mouth tray in a detached configuration;

FIG. 16 is a front perspective view of the mouth tray of the tooth whitening system of FIG. 14;

FIG. 17 is a rear perspective view of the mouth tray of the tooth whitening system of FIG. 14; and

FIG. 18 is a cross-sectional view of the tooth whitening system of FIG. 14.

### DETAILED DESCRIPTION

The following description of the preferred embodiment(s) is merely exemplary in nature and is in no way intended to limit the invention, its application, or uses.

The description of illustrative embodiments according to principles of the present invention is intended to be read in connection with the accompanying drawings, which are to be considered part of the entire written description. In the description of embodiments of the invention disclosed herein, any reference to direction or orientation is merely intended for convenience of description and is not intended in any way to limit the scope of the present invention. Relative terms such as “lower,” “upper,” “horizontal,” “vertical,” “above,” “below,” “up,” “down,” “top” and “bottom” as well as derivatives thereof (e.g., “horizontally,” “downwardly,” “upwardly,” etc.) should be construed to refer to the orientation as then described or as shown in the drawing under discussion. These relative terms are for convenience of description only and do not require that the apparatus be constructed or operated in a particular orientation unless explicitly indicated as such. Terms such as “attached,” “affixed,” “connected,” “coupled,” “interconnected,” and similar refer to a relationship wherein structures are secured or attached to one another either directly or indirectly through intervening structures, as well as both movable or rigid attachments or relationships, unless expressly described otherwise. Moreover, the features and benefits of the invention are illustrated by reference to the exemplified embodiments. Accordingly, the invention expressly should not be limited to such exemplary embodiments illustrating some possible non-limiting combination of features that may exist alone or in other combinations of features; the scope of the invention being defined by the claims appended hereto.

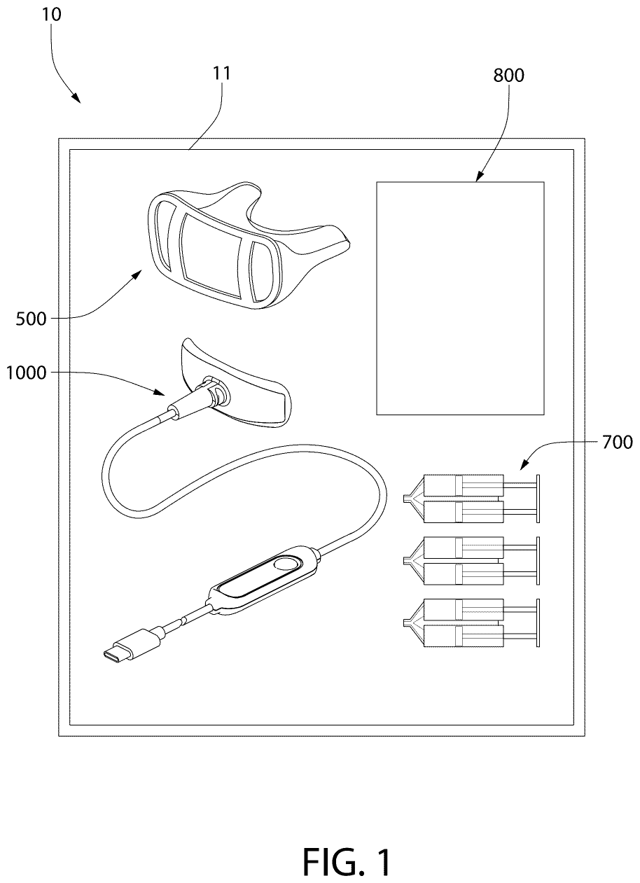

Referring to FIG. 1, a tooth whitening kit 10 is illustrated in accordance with an embodiment of the present invention. The tooth whitening kit 10 may comprise a package 11 containing various components that may be used independently or together to whiten a user's teeth. In some embodiments, the package 11 may be a case. In other embodiments, the package 11 may be a bag, a carton, a blister, a container, or any other style of package typically used to hold multiple components together, including at a point of sale. The package 11 may also provide a way for the end user to transport all of the items of the tooth whitening kit 10 after purchase. In an embodiment, the tooth whitening kit 10 may comprise a whitening device 1000, a mouth tray 500, at least one dispensing unit 700, and a sheet of material 800.

In the exemplified embodiment the at least one dispensing unit 700 comprises three of the dispensing units 700. Any number of the dispensing units 700 may be included in the kit 10 in alternative embodiments. The at least one dispensing unit 700 may comprise a tooth whitening composition that is configured to be applied onto the user's teeth to perform a tooth whitening treatment which may include a light treatment applied by the whitening device 1000. In an embodiment, each of the at least one second dispensing units 700 may be a dual barrel syringe comprising two chambers. In such an embodiment, the tooth whitening composition may comprise a peroxide gel in one chamber and a pH gel in the other chamber. The peroxide gel and the pH gel may be dispensed by the at least one dispensing unit 700 and then mixed together prior to application onto a user's teeth. The tooth whitening composition may comprise any of various amounts and weight percentages of hydrogen peroxide. For example, the tooth whitening composition may comprise 0.1%, 1%, 3%, 6%, 10%, 15%, 20%, 25%, 30%, 40%, 50% hydrogen peroxide, or the like, in various different use cases.

The peroxide gel and the pH gel may be dispensed into a container for mixing prior to application onto a user's teeth. Alternatively, the peroxide gel and the pH gel may be mixed as they are dispensed, and may therefore be dispensed directly onto the mouth tray 500 as discussed further herein below. In some embodiments, applicators may be used to apply the tooth whitening composition directly onto a user's teeth. In other embodiments, the tooth whitening composition may be introduced into channels of the mouth tray and then when the user places their teeth into the channels of the mouth tray the tooth whitening composition may coat the user's teeth. The invention is not intended to be limited by the type of tooth whitening composition used, and various different tooth whitening compositions both those containing hydrogen-peroxide and not, may be used in accordance with the inventions disclosed herein.

In the exemplified embodiment, the tooth whitening composition is described as comprising a peroxide gel and a pH gel. The pH gel acts as a catalyst. In other embodiments, the pH gel may be replaced with other catalysts, including without limitation titanium dioxide, magnesium gluconate, and other heavy or semi-heavy metals.

The sheet of material 800 may be a sheet of ethylene-vinyl acetate in some embodiments. The sheet of material 800 may be formed from other materials that are commonly used to form custom mouth trays in other embodiments. The sheet of material 800 may be used to make a custom mouth tray for a user. The sheet of material 800 may be intended for use by a dental practitioner to form into a proper custom mouth tray for a user, or may be capable of being used by a user without dental practitioner supervision. Specifically, the dental practitioner may make an impression or mold of the user's teeth. The dental practitioner may then place the sheet of material 800 into onto and around the mold of the user's teeth to form the sheet of material 800 into a custom mouth tray that is custom designed to fit the user's teeth. The custom mouth tray may then be used with the whitening device 1000 instead of the mouth tray 500. The inclusion of the sheet of material 800 and the mouth tray 500 in the kit 10 allows each patient and their dental practitioner to decide whether to use the standard mouth tray 500 or to make a custom mouth tray from the sheet of material 800 depending on their preferences or their specific dental health needs. The custom mouth tray 500 may comprise connection features like those described below with reference to the mouth tray 500 to facilitate a coupling between the whitening device 1000 and the custom mouth tray that is formed from the sheet of material 800.

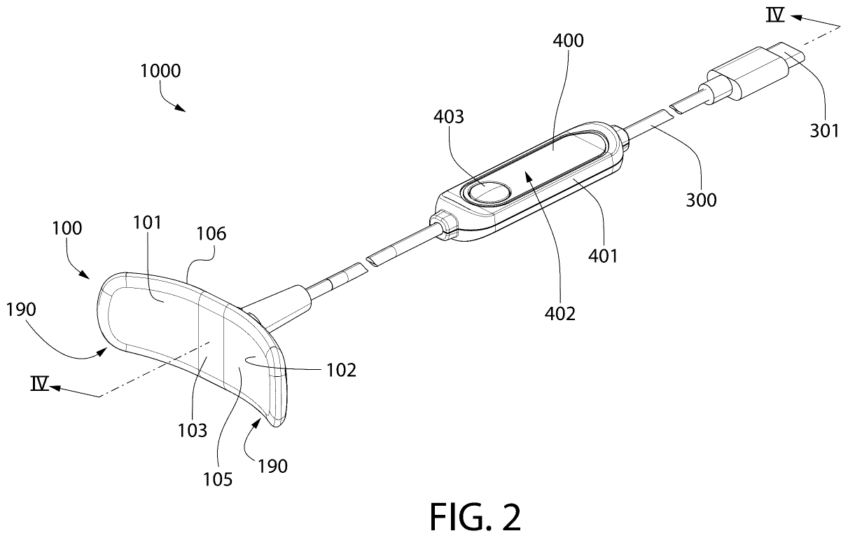

Referring now to FIGS. 2-4, the whitening device 1000 will be described. The whitening device 1000 may be configured to be used within a user's oral cavity to emit electromagnetic radiation or light onto the user's teeth and/or gums to perform or enhance a treatment, such as a whitening treatment, thereon. In one embodiment, the whitening device 1000 may be used in conjunction with a tooth whitening formulation or composition that may be applied onto the teeth prior to enhancement with the whitening device 1000. The tooth whitening formulation or composition may be dispensed from the one or more dispensing units 700, although this is not required and the tooth whitening formulation or composition may be distinct from the kit 10 in some embodiments.

One particular tooth whitening composition which may be used in conjunction with the whitening device 1000 is described in U.S. Pat. No. 11,040,002 . However, the whitening device 1000 may be used alone (i.e., without first coating the teeth with a tooth whitening composition) in some embodiments, and in other embodiments the whitening device 1000 may be used after the user coats the teeth with any tooth whitening composition, such as for example without limitation those containing hydrogen peroxide or carbamide peroxide. The tooth whitening composition which may be used in conjunction with the whitening device 1000 to whiten the teeth is not limited to the specific formulation described in U.S. Pat. No. 11,040,002 and any whitening formulation now known or later discovered which may benefit from light treatment after application to the teeth may be used. Moreover, other formulations, such as anti-sensitivity formulations, fresh breath formulations, or the like which may similarly benefit from a light treatment after application of the formulation to the teeth may be used with the whitening device 1000. One additional example composition is one which includes monoperoxisulfate, which is an oxidizing agent that has a positive reaction with the light being emitted from the device. Furthermore, Vitamin C compounds acting as a reducing agent for stain removal may also be used. In some embodiments, the whitening device 1000 may emit light at wavelengths in the ranges of 380 nm and 440 nm, more specifically between 390 nm and 430 nm, still more specifically between 400 nm and 420 nm, and still more specifically between 400 nm and 420 nm. In some embodiments, the whitening device 1000 may emit light at approximately 410 nm. In some embodiments, the light emitters 164 may have a current of at least 6 mA, or at least 8 mA, or at least 10 mA.

The whitening device 1000 generally comprises a mouthpiece 100, a power cord 300, and a control unit 400. The mouthpiece 100 is the part of the whitening device 1000 that is placed within the user's oral cavity during a treatment procedure. The power cord 300 extends from the mouthpiece 100 and terminates in a plug 301 that may be plugged into a socket of a device which contains a power supply to supply power to the mouthpiece 100, as described herein. In an embodiment, the whitening device 1000 does not have its own power source, but is instead plugged into a socket of another device (which may be an electronic device such as a smart phone, a laptop, a computer, a television, a power bank, an electronic device docking station, or the like or may be a power outlet). Thus, the other device supplies power to the mouthpiece 100 and any electronic components thereof (such as light emitters as described herein) when the plug 301 is engaged with a socket of the electronic device. In the exemplified embodiment, the plug 301 is a USB-C plug. However, the invention is not to be so limited and the plug 301 may take on any structural form desired to enable the power cord 300 to be electronically coupled to an electronic device. Thus, the plug may be a USB Type A, USB Type B, USB 2.0, USB 3.0, USB Mini, USB Micro, USB Micro B, USB Type-C, a standard type plug for engagement with a power outlet of any type depending on geographic location, or the like.

The control unit 400 may be located along the power cord 300. The control unit 400 may be located along the power cord 300 at a position that is closer to the plug 301 than to the mouthpiece 100. This may enhance the comfort to a user during use by not having the weight of the control unit 400 located too close to the mouthpiece 100. As such, the user will not feel the weight of the control unit 400 during use, making it easier to maintain the mouthpiece 100 in the mouth for longer periods of time. The entire whitening device 1000 may be of a very low weight. The weight of the whitening device 1000 may be between 10 g and 50 g, more specifically between 15 g and 30 g, and more specifically between 15 g and 25 g. This enhances the comfort of the whitening device 1000 when in use because the user does not carry much weight in his or her mouth during use.

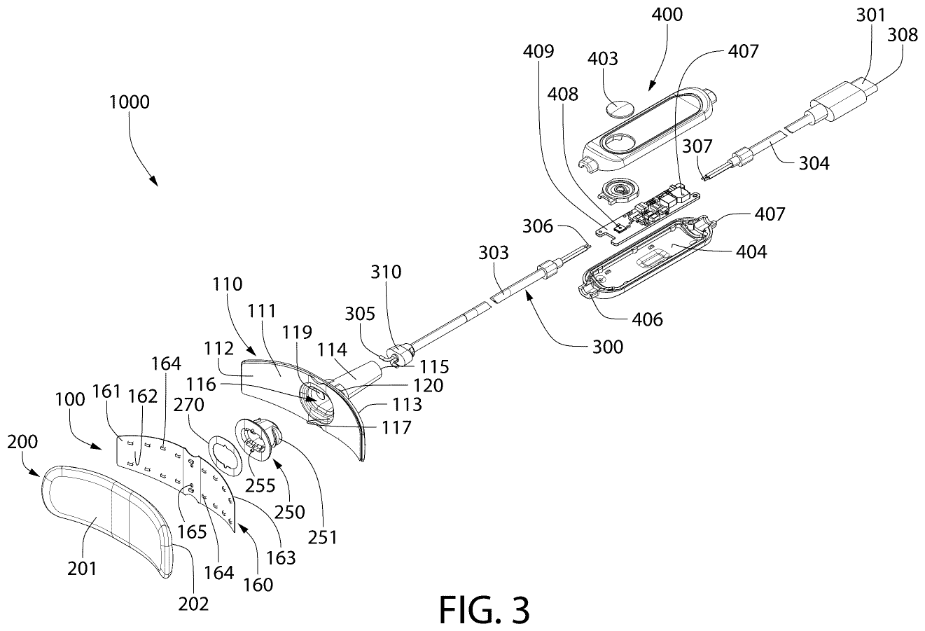

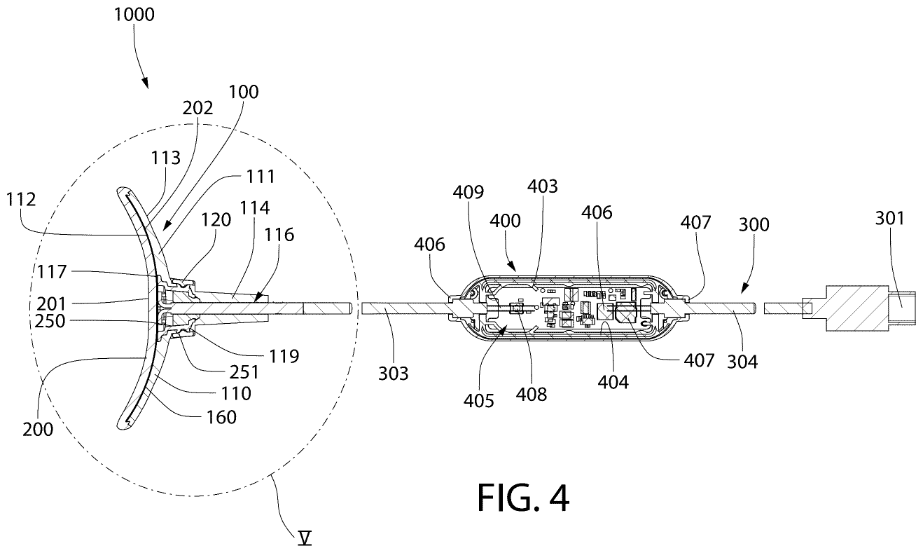

The control unit 400 comprises a housing 401 having an outer surface 402 and an inner surface 404 which defines a cavity 405 (the inner surface 404 and the cavity 405 are shown in FIGS. 3 and 4). There is at least one actuator 403 located along the outer surface 402 of the housing 401. In the exemplified embodiment the at least one actuator 403 is a depressible button that may be used to power the whitening device 1000 on and off and may also be used to modify a mode of operation of the whitening device 1000, as described in greater detail below. The invention is not to be so limited in all embodiments and the at least one actuator 403 may be a slide switch, a conductive switch, a clickable switch, or other implementations in other embodiments. Moreover, while a single actuator 403 is depicted in the drawings, more than one actuator may be used in other embodiments with each actuator performing a different function (on/off and mode selection, for example). The housing 401 may include a display on its outer surface 402 for visually displaying information to a user. The display may display images, text, symbols, light indicators, or the like in some embodiments.

While the at least one actuator 403 may be used to power the whitening device 1000 on and off, it may not function unless the plug 301 of the power cord 300 is electrically coupled to an electronic device, such as any of the types of devices described above. This is because, as noted herein, in certain embodiments the whitening device 1000 may not contain its own power source. This may reduce costs for the whitening device 1000, negate the need for replacement of batteries, and keep the whitening device 1000 at a very low weight. Thus, activating the actuator 403 without the plug 301 being electrically coupled to an electronic device will not result in any powering on of the device. However, when the plug 301 is electrically coupled to an electronic device, activation of the actuator 403 will either change a mode of operation and/or power the device on/off, as described further herein below. In an alternative embodiment, the whitening device 1000 may include an onboard power source to power the whitening device 1000 without having to plug the whitening device 1000 into another power supply (e.g., a mains power supply or an external electronic device).

In the exemplified embodiment, the mouthpiece 100 comprises a front surface 105 and a rear surface 106. The front surface 105 may be concave and the rear surface 106 may be convex. The mouthpiece 100 may further comprise a support member 110, a light emitting assembly 160, and a lens member 200 that are securely and generally non-detachably coupled together so that the mouthpiece 100 is a unitary and integral part of the whitening device 1000 formed by the support member 110 and the lens member 200. The light emitting assembly 160 may be sandwiched between the support member 110 and the lens member 200. That is, while the support member 100, the light emitting assembly 160, and the lens member 200 are separate components, due to the assembly of the mouthpiece 100 the various components cannot be separated from one another, absent tearing or otherwise destroying one or more of the various components. The support member 110 and the lens member 200 may be bonded together during the manufacturing process, such that one of the support member 110 and the lens member 200 may be injection molded onto the other to create a bond between the materials. Alternatively, the lens member 200 and the support member 110 may be adhesively bonded to one another by applying an adhesive material onto the interfacing surfaces (or portions thereof) of the lens member 200 and the support member 110. The support member 110 and the lens member 200 may also have engaging edges and surfaces that interlock the two components together once manufactured to create a mechanical interlock between the two parts.

The support member 110 comprises a support panel 111 having a front surface 112 and a rear surface 113. The support member 110 further comprises a protuberance 114 that extends from the rear surface 113 of the support panel 111 to a distal end 115. The front surface 112 of the support panel 111 may be slightly concave. In other embodiments, the front surface 112 of the support panel 111 may be flat/planar rather than concave, and the support panel 111 may be curved during use due to its flexibility. The support member 110 may be formed from an elastomeric material, such as a thermoplastic elastomer, having a low Shore A hardness which provides the support member 110 with a high degree of flexibility. The support member 110 may be formed from a thermoplastic elastomer having a Shore A hardness between 30 and 40, and more specifically of approximately 35. This is similar to the Shore A hardness of a rubber band, which means that the support member 110 is quite flexible. The thickness of the support member may be between 0.5-2 mm, which adds to the flexibility. One example of a material which may be used to form the support member 110 is MEDALIST®MD-10118 NAT or MEDALIST®MD-10108 NAT sold by Teknor Apex.

As mentioned above, the protuberance 114 protrudes from the rear surface 113 of the support panel 111, which is convex in the exemplified embodiment but could be flat in other embodiments. The protuberance 114 may have a tubular shape and may be conical such that its diameter may taper extending from the support panel 111 to the distal end 115. The specific shape of the protuberance 114 is not to be limiting of the invention in all embodiments and the protuberance 114 may take on other shapes, such as having a polygonal cross-sectional shape rather than a circular cross-sectional shape as in the exemplified embodiment.

The support member 110 may comprise a passageway 116 extending from a first opening 117 in the front surface 112 of the support panel 111 to a second opening 118 in the distal end 115 of the protuberance 114. The passageway 116 may be configured to receive and hold a portion of the power cord 300 while maintaining an end of the power cord 300 in contact with the light emitting assembly 160 so that power can be supplied to light emitters of the light emitting assembly 160, as described further herein.

The support member 110 comprises a first connection feature 119 to facilitate coupling of a strain relief member 250 thereto, as described further below. In the exemplified embodiment, the first connection feature 119 comprises one or more apertures formed through the protuberance 114 of the support member 110 which may receive and/or be engaged by protrusions of the strain relief member 250 to couple the strain relief member 250 thereto.

The light emitting assembly 160 may comprise a flexible sheet 161 and a plurality of light emitters 164. The flexible sheet 161 has a front surface 162 and a rear surface 163. The front surface 162 may be slightly concave and the rear surface 163 may be slightly convex. Alternatively, the front and rear surfaces may be flat/planar such that they are concave and convex only when flexed during use by the user to correspond to the dental arch. The flexible sheet 161 may be a flexible printed circuit board (PCB). The flexible sheet 161 is able to bend, flex, or the like in response to forces being applied thereto. Thus, when the mouthpiece 100 is placed into the user's mouth, the user can flex/bend the entire mouthpiece 100 to match the curve of the user's dental arch.

The plurality of light emitters 164 are coupled to the flexible sheet 161 and are located along the front surface 162 of the flexible sheet 161. The plurality of light emitters 164 may be light emitting diodes (LEDs). However, the invention is not to be so limited in all embodiments and the plurality of light emitters 164 may be other types of light sources in other embodiments. In one embodiment, the light emitters 164 may be formed as printed light emitting diodes, such as those manufactured by Nth Degree. The plurality of light emitters 164 may comprise a plurality of discrete light emitters that are positioned in a spaced apart manner along the front surface 162 of the flexible sheet 161. The plurality of light emitters 164 may protrude from the front surface 162 of the flexible sheet 161 in some embodiments. Although not illustrated, an electrical contact may exist on the rear surface 163 of the flexible sheet 161 so that power from a power source of an external device may be transmitted through the power cable 300 to the plurality of light emitters 164. That is, an exposed electrical conductor 302 of the power cord 300 may be in direct contact with the electrical contact on the rear surface 163 of the flexible sheet 161. The electrical contact on the rear surface 163 of the flexible sheet 161 may in turn be electrically coupled to each of the light emitters 164 (directly to some and indirectly to others, or directly to all) to transmit power to the light emitters 164 so that they may be powered to emit light during a treatment protocol.

The lens member 200 is configured to cover the light emitting assembly 160 so that the front surface 162 of the flexible sheet 161 as well as the light emitters 164 are not exposed to the elements or to the user's oral cavity during use. Rather, the light emitting assembly 160 is sandwiched between the lens member 200 and the support member 110 as discussed herein. Thus, the lens member 200 and the support member 110 may be bonded together to create a hermetically sealed cavity within which the light emitting assembly 160 is located so that the user's saliva and other contaminants may not enter into the cavity and damage the light emitting assembly 160 and other electronic components of the whitening device 1000. The lens member 200 is preferably formed from a thermoplastic elastomer, and may be formed from the same thermoplastic elastomer as the support member 110 (the same material construction, although it may not be the same color). Thus, the lens member 200 may have a Shore A hardness between 30 and 40, and more specifically approximately 35.

Thus, the lens member 200, the light emitting assembly 160, and the support panel 111 of the support member 110 may be flexible, and the entire mouthpiece 100 may be readily flexed in order to match the contours/curvature of a particular user's dental arch or the contours/curvature of the mouth tray 500, as described below. This flexibility of the mouthpiece 100 allows the mouthpiece 100 to be comfortably used by virtually any person regardless of the curvature of that person's dental arch. In an embodiment, the mouthpiece 100 comprises a first side portion 101, a second side portion 102, and a central portion 103 located between the first and second side portions. The first and second side portions 101, 102 may be bent or folded relative to the central portion 103 to such a degree that the front surface 201 of the lens member 200 along the first and second side portions 101, 102 may contact the front surface 201 of the lens member 200 along the central portion 103. Of course, in other embodiments the flexibility may be modified, but the first and second side portions 101, 102 may generally be capable of being bent or folded at least 90° relative to the central portion. In an embodiment, the first and second side portions 101, 102 may be folded relative to the central portion 103 so that the first and second side portions 101 form an angle between at least 60° and 90° relative to the central portion.

In an embodiment, the first and second side portions 101, 102 may also be bent/folded rearwardly relative to the central portion 103 so that the rear surface 113 of the support member 110 along the first and second side portions 101, 102 may contact the rear surface 113 of the support member 110 along the central portion 103. Thus, the first and second side portions 101, 102 may be folded approximately 180° relative to the central portion 103 in either direction. This is an incredible amount of flexibility which allows the mouthpiece 100 to readily fit the contours of virtually any human oral cavity dental arch.

The lens member 200 may be formed from a clarified thermoplastic elastomer. Stated another way, the lens member 200 may be transparent. As such, any light emitted by the light emitters 164 will readily pass through the lens member 200 and onto the user's teeth. The lens member 200 may be fully transparent to the light being emitted by the light emitters 164 (which might be light having a wavelength between 390 nm and 430 nm, more specifically between 400 nm and 420 nm, and still more specifically approximately 410 nm, approximately in this context meaning plus or minus 5 nm). It is worth noting that the support member 110 may be formed from an opaque color, such as white, red, green, blue, black, or the like, or the support member 110 may also be transparent if so desired. However, the support member 110 need not be transparent in all embodiments because the light emitted by the light emitters 164 is not intended to transmit through the support member 110. In some embodiments, the support member 110 may be transparent so that the light emits therethrough, to provide for an aesthetic effect. The lens member 200 may include facets on its front and/or rear faces to help with the dispersion of the light emitted by the light emitters 164. The lens member 200 may be semi-transparent in some embodiments. The lens member 200 may comprise textures or other features that facilitate the dispersion of the light generated by the light emitters 164.

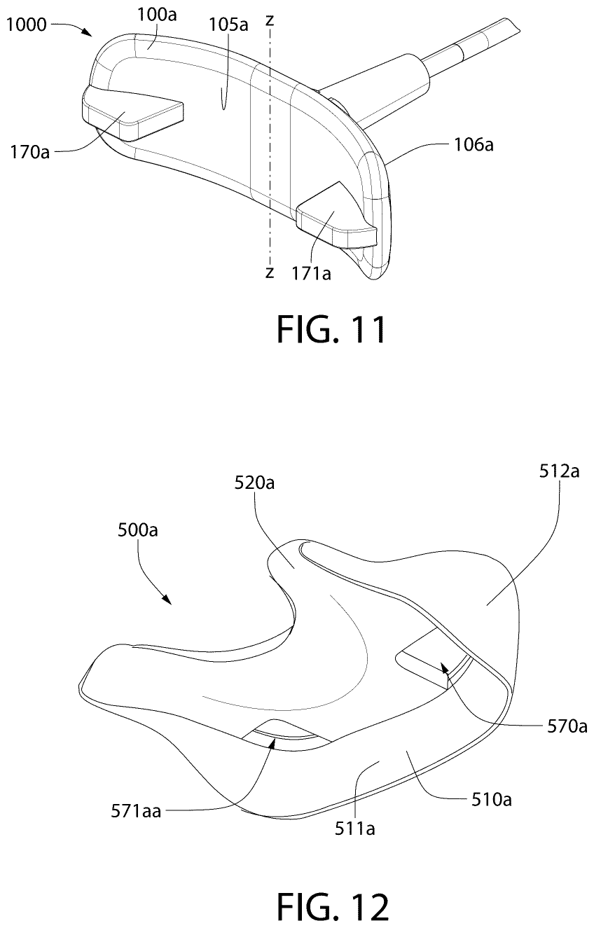

The lens member 200 comprises a front surface 201 and a rear surface 202. The front surface 201 may be concave and the rear surface 202 may be convex. Of course, as with the other components of the mouthpiece 100, the front and rear surfaces 201, 202 may be flat/planar in other embodiments and the curvature may only be achieved when the device is placed in the user's oral cavity. The lens member 200 may comprise one or more bite protrusions extending from the front surface 201 of the lens member 200. An example of this is shown in FIG. 11 and will be described below.

The whitening device 1000 further comprises the strain relief member 250 (mentioned above) and a double-sided adhesive panel 270. The strain relief member 250 is positioned within the passageway 116 of the support member 110 to facilitate locking the power cord 300 into position and preventing detachment of the power cord 300 from the mouthpiece 100. The strain relief member 250 comprises a second connection feature 251 that engages the first connection feature 119 of the support member 110 to lock the strain relief member 250 to the support member 110 within the passageway 116. In the exemplified embodiment, the second connection feature 251 is a protrusion that extends into/nests within the aperture of the first connection feature 119 to lock the strain relief member 250 into place. Thus, the second connection feature 251 of the strain relief member 250 may be exposed through the aperture of the first connection feature 119 of the support member 110. The strain relief member 250 may be formed from a hard plastic material (ABS, polypropylene, or similar hard plastics), metal, or other rigid materials.

The strain relief member 250 comprises at least one protrusion 255 protruding from its front surface. In the exemplified embodiment, there are two of the protrusions 255, but there could be just one or more than two in other embodiments. The light emitting assembly 160 comprises at least one hole 165. In the exemplified embodiment, the light emitting assembly 160 comprises two of the holes 165. In the exemplified embodiment, the protrusions 255 of the strain relief member 250 extend into and through the holes 165 in the light emitting assembly 160. Thus, because the strain relief member 250 is secured in place within the passageway 116 and is fixed/non-movable relative to the support member 110, the engagement of the protrusions 255 of the strain relief member 250 with the holes 165 in the light emitting assembly 160 serves to hold the light emitting assembly 160 in place and prevent the light emitting assembly 160 from moving within the space between the support member 110 and the lens member 200.

The whitening device 1000 may be assembled in the following order. First, the strain relief member 250 is positioned within the passageway 116 of the support member 210. This may be achieved by sliding the strain relief member 250 into the passageway 116 from the opening 117 in the front surface 112 of the support panel 111, or by injection molding the support member 110 directly onto and around the strain relief member 250. Next, the power cord 300 (or a portion thereof) is slid through the through-hole of the strain relief member 250 in a direction moving from the front surface 112 of the support panel 111 to the rear surface 113 of the support panel 111. Next, the adhesive panel 270 may be adhered to the front surface of the strain relief member 250 (although this step may be omitted). Next, the light emitting assembly 160 may be positioned with its rear surface 163 abutting the front surface 112 of the support panel 111. The adhesive panel 270 may serve to hold the light emitting assembly 160 in this position. The light emitting assembly 160 may be positioned so that the electrical conductor 302 of the power cord 300 is in contact with an electrical contact on the rear surface 163 of the light emitting assembly 160. Finally, the lens member 200 may be injection molded onto the light emitting assembly 160 and support panel 111 to form the mouthpiece 100. At this point, the light emitting assembly 160 is sandwiched between the support panel 111 and the lens member 200 and the power cord 300 is locked in place with a portion of the power cord 300 located within the passageway 116 and another portion of the power cord 300 protruding from the distal end 115 of the protuberance 114 of the support member 110. Alternatively, the lens member 200 may be separately formed and then bonded/attached to the support member 110 using adhesives or the like.

As best shown in FIG. 3, the power cord 300 may comprise a first cord portion 303 and a second cord portion 304, with the control unit 400 being located between the first and second cord portions 303, 304. The first cord portion 303 extends from a first end 305 to a second end 306 and the second cord portion 303 extends from a first end 307 to a second end 308. The first end 305 of the first cord portion 302 comprises the electrical conductor 302. The second end 306 of the first cord portion 302 is coupled to a proximal end 406 of the control unit 402. The second end 308 of the second cord portion 303 comprises the plug 301. The first end 307 of the second cord portion 303 is coupled to a distal end 407 of the control unit 402.

In the exemplified embodiment, the first cord portion 303 has a first length measured from the first end 305 to the second end 306 and the second cord portion 304 has a second length measured from the first end 307 to the second end 308. The first length may be greater than the second length. In some embodiments, the first length may be at least two times the second length. In some embodiments, the power cord 300 may have a length, and the control unit 400 may be located along a distal-one third of the length of the power cord 300. The power cord 300 may have a length between 30 inches and 40 inches, and more specifically between 32 inches and 36 inches. In such embodiment, the control unit 400 may be located no more than 15 inches from the plug 301. In other embodiments, the control unit 400 may be located no more than 12 inches from the plug 301. In some embodiments, the control unit 400 may be located between 8 inches and 12 inches from the plug 301. Thus, the control unit 400 is located closer to the second end 308 of the second cord portion 304 than to the first end 305 of the first cord portion 303. Or, as noted above, the control unit 400 is located closer to the plug 301 than to the mouthpiece 100. This ensures that the user is not supporting the weight of the control unit 400 when the mouthpiece 100 is in the user's oral cavity, which may occur if the control unit 400 were located closer to the mouthpiece 100 than to the plug 301.

The control unit 400 comprises the actuator 403 and other circuitry and/or electronic components operably coupled to the actuator 403 and located within the cavity 405 of the housing 401. The actuator 403 may comprise or be operably coupled to a switch 408 that is located on a printed circuit board 409. Furthermore, the control unit 400 may comprise a processor 406 and a timer 407 that are operably coupled together and to the switch 408. Additional electronic components which may be necessary for the operation and function of the whitening device 1000 may also be included on the printed circuit board 409 housed within the cavity 405 of the housing 401. The processor 406 and the timer 407 may be located on the printed circuit board 409. Thus, when the power cord 300 is plugged in and the user activates the actuator 403, the processor may cause the light emitters 164 of the light emitting assembly 160 to emit for a predetermined period of time, as determined by the timer 407. The predetermined period of time may be preset in some embodiments, and it may be adjustable in other embodiments. That is, a user may be able to adjust or otherwise change the ON time for the light emitters 164 for a particular tooth whitening session.

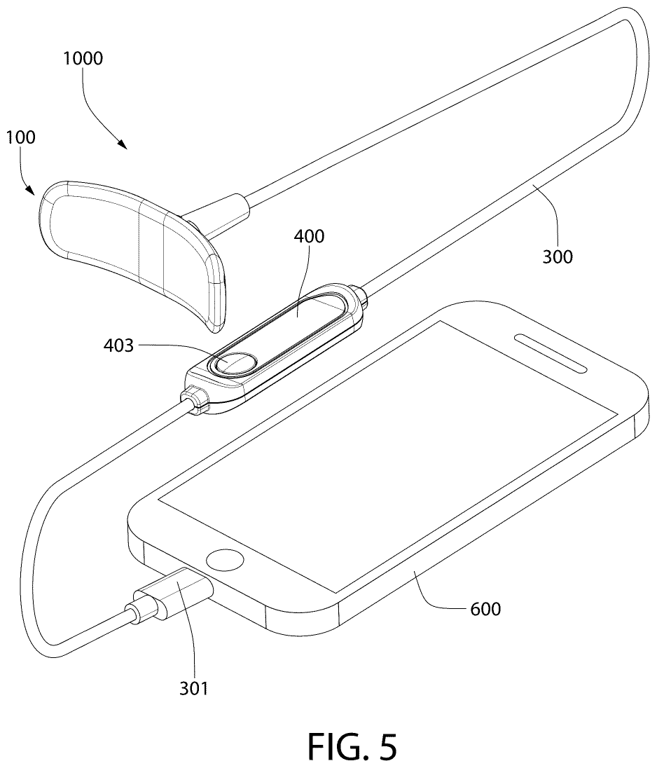

Referring to FIG. 5, the oral cavity treatment device 1000 is illustrated coupled to an electronic device 600. Specifically, the plug 301 of the power cord 300 is plugged into a socket of the electronic device 600 so that power may be supplied from the electronic device 600 to the oral cavity treatment device 1000 to power on the light emitters 164 as described herein. With the plug 301 of the power cord 300 plugged into the socket of the electronic device 600, a user can actuate the actuator 403 on the control unit 400 to initiate a treatment session. Actuation of the actuator 403 may result in the light emitters 164 illuminating for a predetermined period of time as determined by the timer, processor, or the like of the control unit 400. In the exemplified embodiment, the electronic device 600 is a smart phone. As discussed above, the invention is not to be so limited in all embodiments and the electronic device 600 may take on other forms such as being any of the types of devices or power supply sources described above.

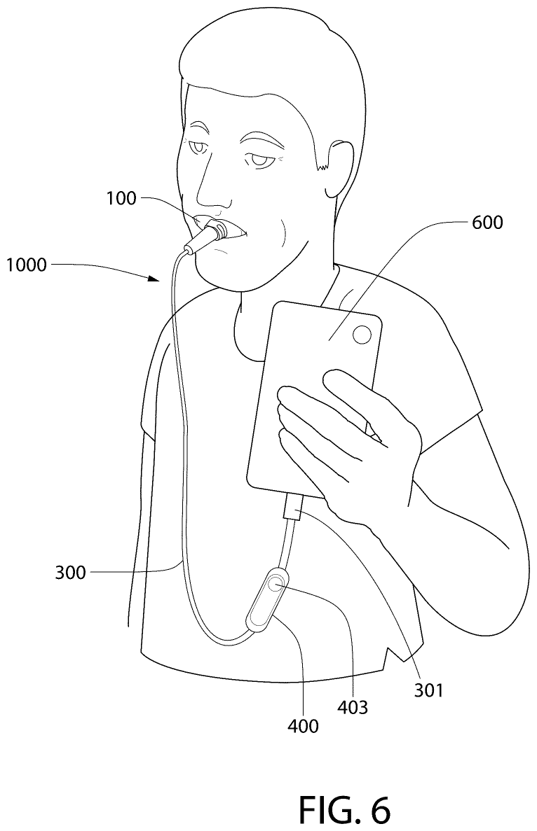

Referring to FIG. 6, the plug 301 of the power cord 300 is plugged into the electronic device 600 and the mouthpiece 100 of the whitening device 1000 is positioned within the user's oral cavity. The user then activates the actuator 403 to start the treatment protocol. Actuating the actuator 403 may cause the light emitters 164 to emit light for a predetermined period of time, as discussed herein. The predetermined period of time may be 5 minutes, 10 minutes, 15 minutes, 20 minutes, 25 minutes, 30 minutes, or any other desired time period. The power cord 300 has a length of between 30 and 40 inches, or between 32 and 36 inches, as described above. This length should be sufficient to enable the user to store their phone in a rear pocket of their pants while the mouthpiece 100 is positioned within their mouth. In some embodiments, the power cord 300 may be coupled to an extension cord that is plugged into a power source, for example when the device is used in a dental office.

In an embodiment, a tooth whitening composition may be applied onto the front surfaces of the user's teeth prior to inserting the mouthpiece 100 of the whitening device 1000 into the user's mouth. Specifically, the tooth whitening composition may be applied directly onto the front surfaces of the user's “smile” teeth (the teeth which are visible when the user smiles) prior to inserting the mouthpiece 100 into the user's mouth. Then, the mouthpiece 100 may be inserted into the user's mouth and activated to transmit light onto the surfaces of the user's teeth that are pre-coated with the tooth whitening composition. The combination of whitening composition and light treatment may enhance the whitening benefit for the user and generates a greater change in tooth color in a shorter period of time than when using a whitening composition only.

Referring briefly to FIG. 2, the mouthpiece 100 of the whitening device 1000 may comprise one or more first connection features 190 that are configured to facilitate an attachment of the mouthpiece 100 to the mouth tray 500. In an embodiment, the one or more first connection features 190 may comprise a first connection feature and a second connection feature. The first connection feature may comprise the first side portion 101 of the mouthpiece 100. In an embodiment, at least a portion of the first side portion 101 of the mouthpiece 100 forms the first connection feature of the one or more first connection features. The second connection feature may comprise the second side portion 102 of the mouthpiece 100. In an embodiment, at least a portion of the second side portion 102 of the mouthpiece 100 forms the second connection feature of the one or more first connection features. In an alternative embodiment, one or more bite protuberances of the mouthpiece 100 may form the first and second connection features (see, for example, FIG. 11, described below).

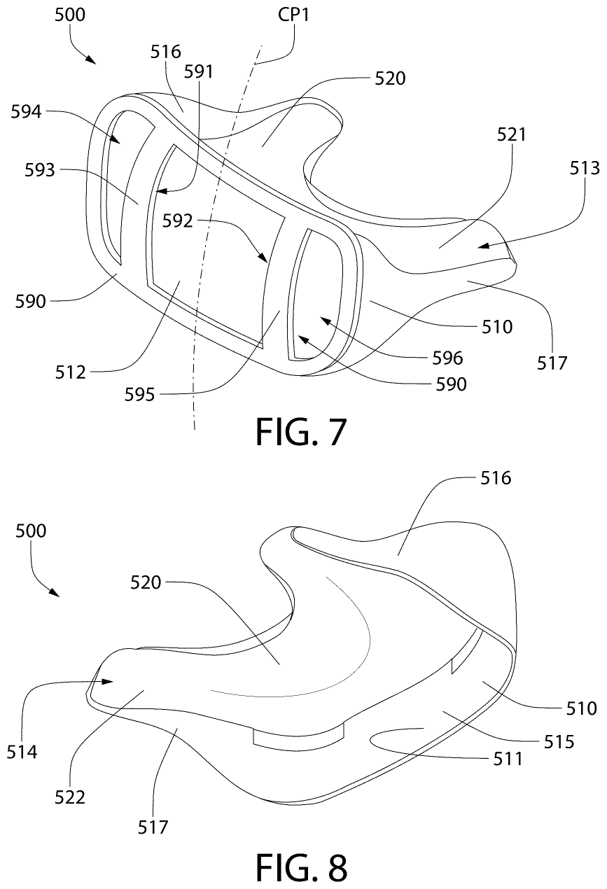

Referring to FIGS. 7 and 8, the mouth tray 500 will be described in accordance with an embodiment of the present invention. The mouth tray 500 may be configured to be inserted into the user's mouth during a whitening or other oral care treatment session. Thus, the mouth tray 500 may be formed from a material that is safe to place into the user's mouth. The mouth tray 500 may be formed from a hard plastic material, an elastomeric material, or the like in various different embodiments. The mouth tray 500 may be formed from a transparent material in some embodiments. The mouth tray 500 may be formed from a light transmissive material. The mouth tray 500 may be formed from a material that allows light generated by the light emitters 164 of the whitening device 100 to pass through the mouth tray 500 and onto the user's teeth during a treatment session.

The mouth tray 500 may comprise a support wall 510 having a front surface 511 and a rear surface 512, a bite platform 520 extending from the front surface 511 of the support wall 510, and one or more second connection features 590 located on the rear surface 512 of the support wall 510. The front surface 511 of the support wall 510 may be concave and the rear surface 512 of the support wall 510 may be convex or planar. The support wall 510 may be arcuate and may include a rear wall section 515, a first side wall section 516, and a second side wall section 517. The bite platform 520 may comprise an upper surface 521 and a lower surface 522. In an embodiment, the mouth tray 500 comprises an upper teeth channel 513 defined by the upper surface 521 of the bite platform 520 and an upper portion of the front surface 511 of the support wall 510. In an embodiment, the mouth tray 500 comprises a lower teeth channel 514 defined by the lower surface 522 of the bite platform 520 and a lower portion of the front surface 511 of the support wall 510. The upper teeth channel 513 may be configured to receive the user's upper teeth during a treatment session. The lower teeth channel 514 may be configured to receive the user's lower teeth during a treatment session.

The mouth tray 500 may comprise a central reference plane CP1. The mouth tray 500 may be symmetrical about the central reference plane CP1. In the exemplified embodiment, the one or more second connection features 590 comprises a pair of the second connection features 590. Specifically, the one or more second connection features 590 comprises a first capture element 591 located on a first side of the central reference plane CP1 and a second capture element 592 located on a second side of the central reference plane CP1. In the exemplified embodiment, the first capture element 591 comprises a first strap portion 593 that extends over the rear surface 512 of the support wall 510 in a spaced apart manner from the rear surface 512 of the support wall 510 and a first receptacle 594 that is defined between the first strap portion 593 and the rear surface 512 of the support wall 510. In the exemplified embodiment, the second capture element 592 comprises a second strap portion 595 that extends over the rear surface 512 of the support wall 510 in a spaced apart manner from the rear surface 512 of the support wall 510 and a second receptacle 596 that is defined between the second strap portion 595 and the rear surface 512 of the support wall 510.

In the exemplified embodiment, each of the first and second strap portions 593, 595 extends vertically from or near a top of the support wall 510 to at or near a bottom of the support wall 510. In the exemplified embodiment, each of the first and second strap portions 593, 595 are spaced inwardly from the side edges of the support wall 510. In other embodiments, the first and second strap portions 593, 595 may extend to the side edges of the support wall 510 while still defining the first and second receptacles 594, 596, respectively.

In some embodiments, the first and second strap portions 593, 595 may be formed from a material which differs from the material of the mouthpiece 100 to avoid high friction between the mouthpiece 100 and the strap portions 593, 595 during attachment of the mouthpiece 100 to the mouth tray 500. Such friction, if the mouthpiece 100 and the strap portions 593, 595 are formed from the same material, may create difficulty for the user in achieving the attachment. The first and second strap portions 593, 595 may be formed from a hard plastic material, such as polypropylene, polyethylene, polyvinyl chloride, acrylonitrile butadiene styrene, or the like.

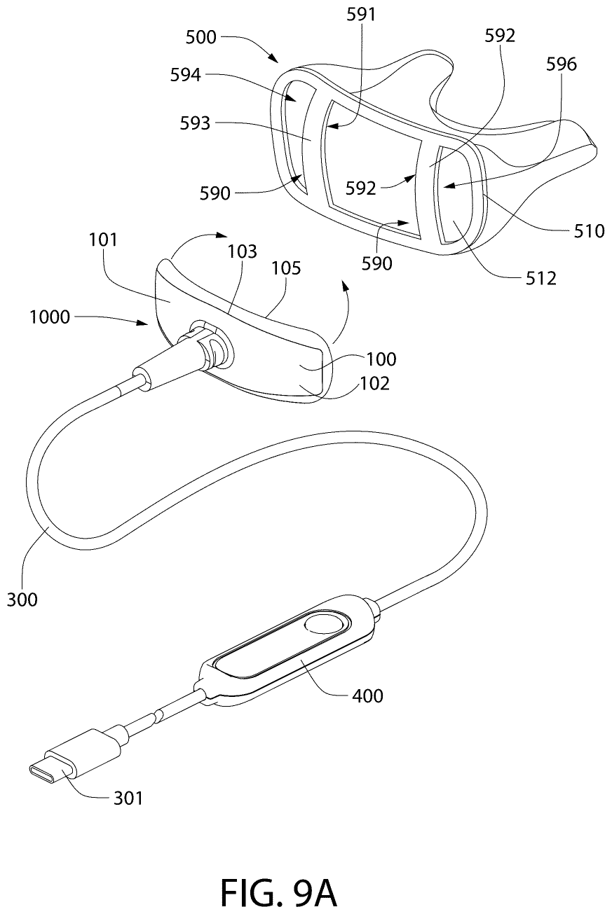

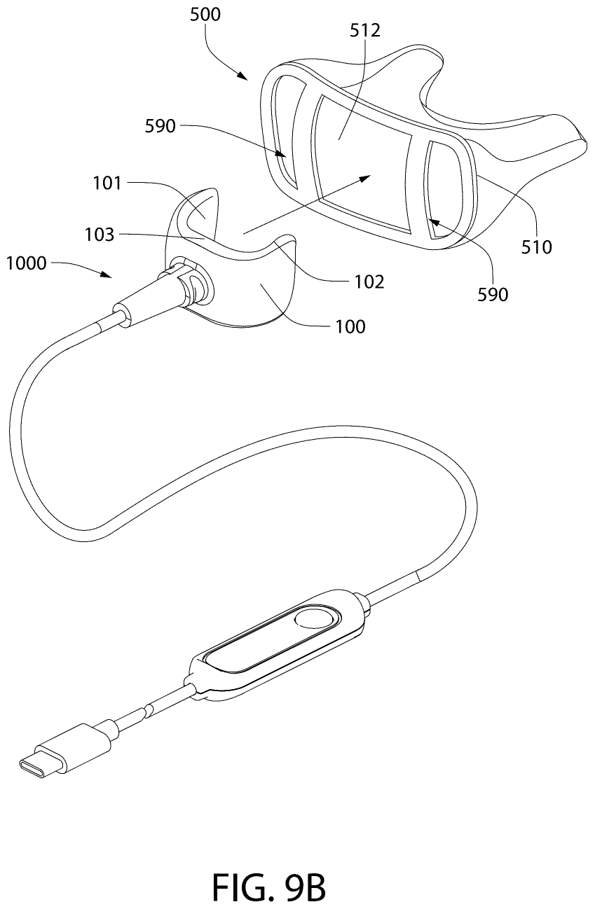

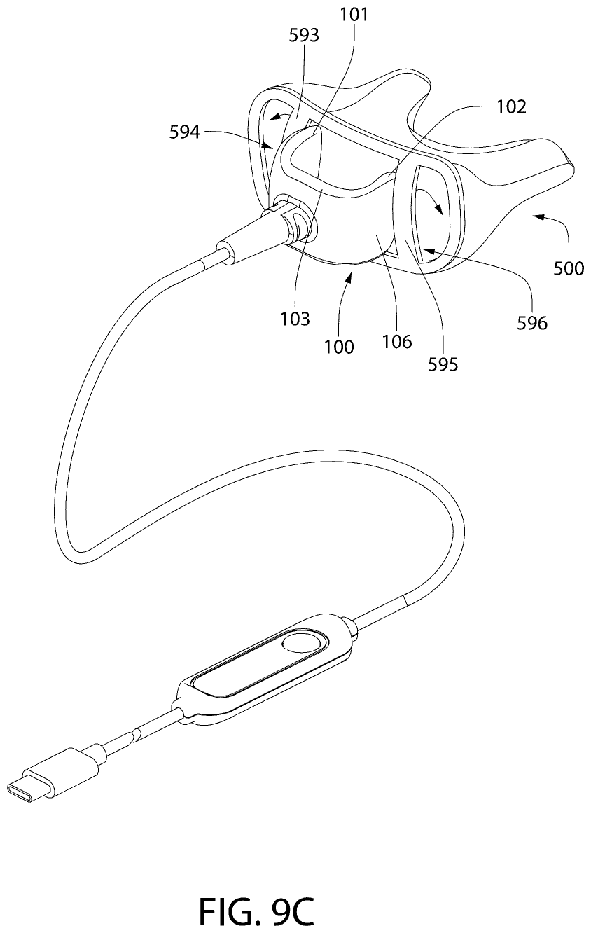

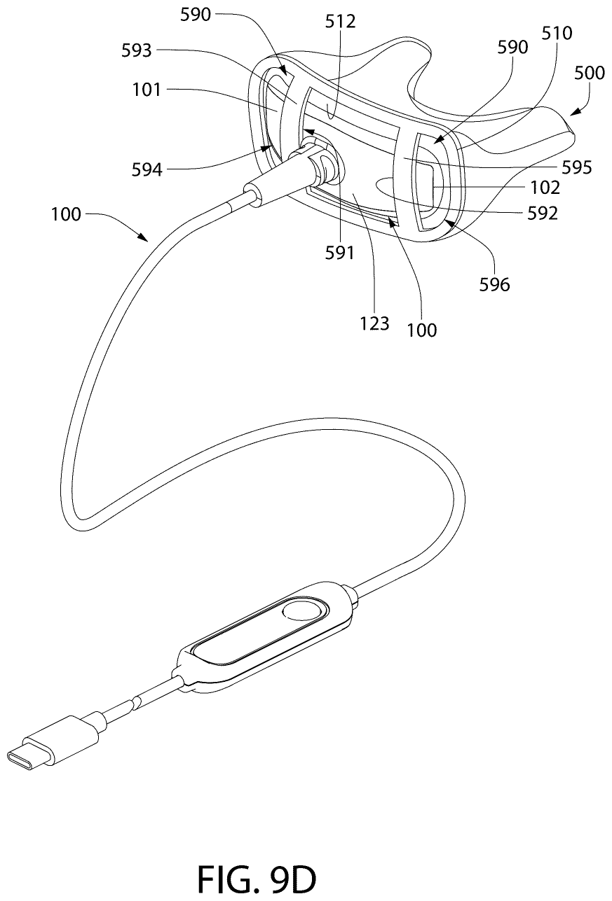

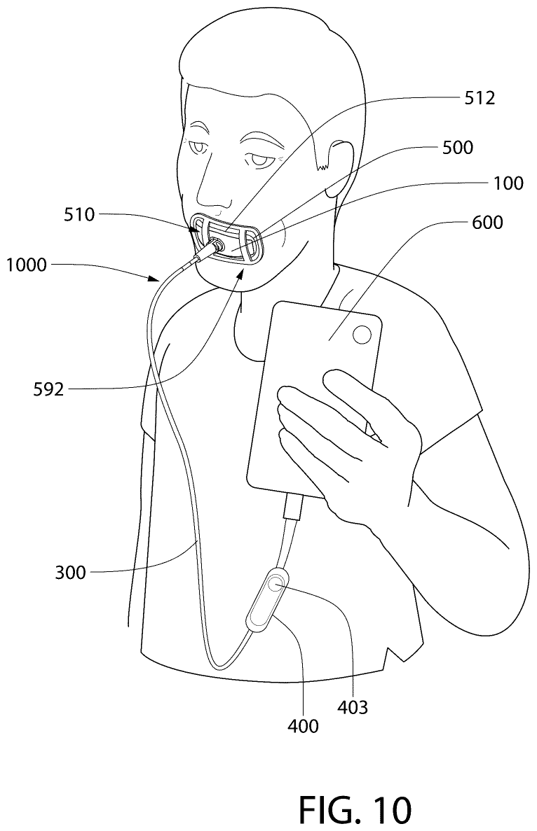

Referring sequentially to FIGS. 9A-9D, the coupling of the whitening device 1000 to the mouth tray 500 will be described in accordance with an embodiment of the present invention. The whitening device 1000 may be detachably coupled to the mouth tray 500 via engagement between the one or more first connection features 190 of the mouthpiece 100 of the whitening device 1000 and the one or more second connection features 590 of the mouth tray 500. In an embodiment, the whitening device 1000 and the mouth tray 500 may be repetitively coupled together and detached from one another. In an embodiment, the whitening device 1000 may be used either without the mouth tray 500 (see, for example, FIG. 6) or with the mouth tray 500 detachably coupled thereto (see, for example, FIG. 10).

Referring to FIG. 9A, the whitening device 1000 is illustrated with the front surface 105 of the mouthpiece 100 facing the rear surface 512 of the support wall 510 of the mouth tray 500. When the mouthpiece 100 is coupled to the mouth tray 500, the front surface 105 of the mouthpiece 100 is adjacent to and facing the rear surface 512 of the support wall 510. Furthermore, the light emitters 164 of the whitening device 1000 are configured to generate light that is emitted from the front surface 105 of the mouthpiece 100. Thus, the light will be emitted from the front surface 105 of the mouthpiece 100 and through the support wall 510 of the mouth tray 500 and onto the desired surfaces of the user's oral cavity, such as the front surfaces of the user's “smile” teeth (i.e., the teeth which are visible to others when the user is smiling). As such, when preparing to attach the whitening device 1000 to the mouth tray 500, the front surface 105 of the mouthpiece 100 faces the rear surface 512 of the support wall 510 of the mouth tray 500.

Next, referring to FIG. 9B, the first side portion 101 of the mouthpiece 100 is folded or bent relative to the central portion 103 of the mouthpiece 100 and the second side portion 102 of the mouthpiece 100 is folded or bent relative to the central portion 103 of the mouthpiece 100. The first and second side portions 101, 102 may be folded or bent forwardly such that the front surface 105 of the mouthpiece 100 along the first and second side portions 101, 102 are folded towards the front surface 105 of the mouthpiece 100 along the central portion 103. The mouthpiece 100 may be bent or folded so that the radius of curvature of the front surface 105 is decreased relative to its natural or biased state. In an embodiment, the mouthpiece 100 may be formed from or may comprise a flexible wall. The flexible wall may comprise the front and rear surfaces 105, 106 of the mouthpiece 100. The flexible wall may further comprise the first and second side portions 101, 102 and the central portion 103 of the mouthpiece 100.

Referring to FIGS. 9C and 9D concurrently, with the first and second side portions 101, 102 of the mouthpiece 100 folded as shown in FIG. 9B, the mouthpiece 100 is moved towards the rear surface 512 of the support wall 510 of the mouth tray 500. The first side portion 101 of the mouthpiece 100 is then inserted into the first receptacle 594 of the first capture element 591 and the second side portion 102 of the mouthpiece 100 is inserted into the second receptacle 596 of the second capture element 591. While inserting the first and second side portions 101, 102 of the mouthpiece 100 into the first and second receptacles 594, 596, respectively, the user will allow the first and second side portions 101, 102 to become unfolded by ceasing the application of force thereon to cause the folding of the first and second side portions 101, 102 relative to the central portion 103 as discussed above. Specifically, the mouthpiece 100 has a biased configuration when no user force is applied thereon, which is the configuration shown in FIG. 2. The mouthpiece 100 is folded into the configuration shown in FIG. 9B by a user applying a force onto the first and second side portions 101, 102 of the mouthpiece 100 relative to the central portion 103 of the mouthpiece 100. When the user releases the force, the first and second side portions 101, 102 will unflex or unfold, returning to the orientation of the mouthpiece 100 as shown in FIG. 2. As the first and second side portions 101, 102 unfold, they will pass into the first and second receptacles 594, 596, as described.

Once so positioned, the first strap portion 593 of the first capture element 591 of the one or more second connection features 590 extends across the rear surface 106 of the mouthpiece 100. Thus, the first side portion 101 of the mouthpiece 10 nests within the first receptacle 594 of the first capture element 591 of the one or more second connection features 590 of the mouth tray 500 and the first strap portion 593 of the first capture element 591 extends across the rear surface 106 of the first side portion 101 of the mouthpiece 100. Similarly, the second side portion 102 of the mouthpiece 100 nests within the second receptacle 596 of the second capture element 592 of the one or more second connection features 590 of the mouth tray 500. Thus, the second side portion 102 of the mouthpiece 101 nests within the second receptacle 596 of the second capture element 592 of the one or more second connection features 590 of the mouth tray 500 and the second strap portion 595 of the second capture element 592 extends across the rear surface 106 of the second side portion 102 of the mouthpiece 100. The first and second side portions 101, 102 of the mouthpiece 101 are thereby retained between the first and second strap portions 593, 595 (respectively) and the rear surface 512 of the support wall 510 of the mouth tray 500.

FIG. 9D illustrates the mouthpiece 100 of the whitening device 1000 coupled to the mouth tray 500. The mouthpiece 100 may be detached from the mouth tray 500 by pulling the mouthpiece 100 away from the mouth tray 500 with a sufficient force to cause the mouthpiece 100 to bend to retract the first and second portions 101, 102 of the mouthpiece 100 from the first and second receptacles 594, 596, respectively. This process may be repeated as desired for subsequent treatments. Thus, the whitening device 1000 may be used by placing the mouthpiece 100 directly into the user's mouth without the mouth tray 500 (see, for example, FIG. 6), or the whitening device 1000 may be used by placing the mouth tray 500 into the user's mouth while the mouthpiece 100 is coupled to the mouth tray 500, as shown in FIG. 10 described below.

Referring to FIG. 10, the tooth whitening system is illustrated whereby the mouthpiece 100 of the whitening device 1000 is coupled to the mouth tray 500, and the mouth tray 500 is positioned in the user's mouth to perform a treatment. In this use scenario, the tooth whitening composition may be introduced into the upper and lower teeth channels 513, 514 of the mouth tray 500 prior to inserting the mouth tray 500 into the user's mouth. Then, when the user's upper teeth come to rest within the upper teeth channel 513 and the user's lower teeth come to rest within the lower teeth channel 514, the tooth whitening composition will coat the user's teeth. Alternatively, the tooth whitening composition could be applied directly to the teeth prior to inserting the mouth tray 500 into the user's mouth.

As discussed previously, the mouthpiece 100 is coupled to the mouth tray 500 so that the front surface 105 of the mouthpiece 100 faces the rear surface 512 of the support wall 510 of the mouth tray 500. This ensures that the light emitted by the light emitters is emitted from the front surface 105 of the mouthpiece 100 and through the support wall 510 and onto the user's teeth. In FIG. 10, the mouthpiece 100 and the rear surface 512 of the support wall 510 of the mouth tray 500 are visible. This is to depict the attachment of the mouthpiece 100 to the mouth tray 500. It is possible that during a normal use the mouthpiece 100 and the rear surface 512 of the support wall 510 may be located within the user's mouth and hidden from view behind the user's lips.

Referring to FIG. 11, an alternative embodiment of a mouthpiece 100a of the whitening device 1000 of FIG. 1 is illustrated. The mouthpiece 100a is very similar to the mouthpiece 100 and the description of the mouthpiece 100 above is applicable except for the additional features associated with the mouthpiece 100a described below. That is, the mouthpiece 100a may include every feature of the mouthpiece 100 described above and some additional features which will be described below.

The mouthpiece 100a comprises a front surface 105a and a rear surface 106a. The mouthpiece 100a may be arcuate. Specifically, the front surface 105a of the mouthpiece 105a may be concave and the rear surface 106a of the mouthpiece 100a may be convex. In this embodiment, the mouthpiece 100a may comprise a first bite protuberance 170a protruding from the front surface 105a of the mouthpiece 100a and a second bite protuberance 171a protruding from the front surface 105a of the mouthpiece 100a. In the exemplified embodiment, the first and second bite protuberances 170a, 171a are spaced apart along the front surface 105a of the mouthpiece 100a. In the exemplified embodiment, the first and second bite protuberances 170a, 171a are located on opposite sides of a vertical centerline Z-Z of the mouthpiece 100a. In an alternative embodiment, the mouthpiece 100a may comprise just one continuous bite protuberance protruding from the front surface 105a.

In the exemplified embodiment, the one or more first connection features of the mouthpiece 100a of the whitening device 100 may comprise the first bite protuberance 170a and the second bite protuberance 171a. Thus, the first and second bite protuberances 170a, 171a may be used as the structures that facilitate the attachment of the mouthpiece 100a to the mouth tray.

In that regard, FIG. 12 illustrates an alternative embodiment of a mouth tray 500a. The mouth tray 500a is very similar to the mouth tray 500 and the description of the mouth tray 500 above is applicable except for the additional features associated with the mouth tray 500a described below. That is, the mouth tray 500a may include every feature of the mouth tray 500 described above and some additional features which will be described below.

The mouth tray 500a comprises a support wall 510a having a front surface 511a and a rear surface 512a and a bite platform 520a protruding from the front surface 511a of the support wall 510a. In this embodiment, the mouth tray 500a further comprises a first recess 570a extending through the support wall 510a and into a rear portion of the bite platform 520a and a second recess 571a extending through the support wall 510a and into the rear portion of the bite platform 520a. The first and second recesses 570a, 571a may be spaced apart from one another. In one alternative embodiment, instead of two recesses there may be just one recess. In particular, the number of the recesses should correspond with the number of the bite protuberances 170a, 171a, of the mouthpiece 100a since they will mate/engage with one another to facilitate the coupling of the mouthpiece 100a to the mouth tray 500a. In this embodiment, the one or more second connection features of the mouth tray 500a may comprise the first and second recesses 570a, 571a.

In the exemplified embodiment, the first and second recesses 570a, 571a form holes that extend through the bite platform 520a from the upper surface of the bite platform 520a to the lower surface of the bite platform 520a. In other embodiments, the first and second recesses 570a, 570b may not extend through the full thickness of the bite platform 520a but may instead be open only at one of the upper and lower surfaces of the bite platform 520a, but not both. In still other embodiments, the first and second recesses 570a, 571a may be embedded within the bite platform 520a so that the first and second recesses 570a, 57a have only one opening in the rear surface 512a of the support wall 510a with no openings in the upper and lower surfaces of the bite platform 520a.

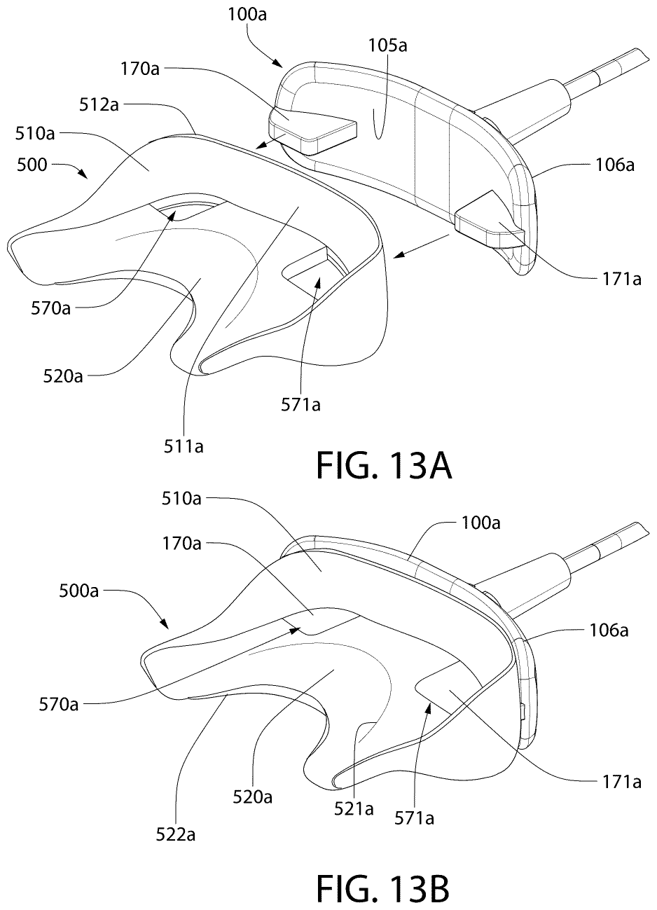

Referring to FIGS. 13A and 13B, the coupling of the mouthpiece 100a to the mouth tray 500a will be described. First, as shown in FIG. 13A, the mouthpiece 100a is positioned so that the front surface 105a of the mouthpiece 100a is adjacent to the rear surface 512a of the support wall 510a of the mouth tray 500a. The mouthpiece 100a is further positioned so that each of the first and second bite protuberances 170a, 171a of the mouthpiece 100a is aligned with one of the first and second recesses 570a, 571a of the mouth tray 500a. Next, as shown in FIG. 13B, the mouthpiece 100a is moved towards the mouth tray 500a until the first bite protuberance 170a enters the first recess 570a and the second bite protuberance 171a enters the second recess 571a. In an embodiment, the first and second bite protuberances 170a, 171a may fit within the first and second recesses 570a, 571a with a tight fit or friction fit or interference fit, which therefore maintains the coupling of the mouthpiece 100a to the mouth tray 500a until detached by a user pulling the mouthpiece 100a and the mouth tray 500a apart.

In another embodiment, the first and second bite protuberances 170a, 171a may fit somewhat loosely in the first and second recesses 570a, 571a but without a tight fit or friction fit or interference fit. In such embodiment, the engagement of the first and second bite protuberances 170a, 171a with the first and second recesses 570a, 571a will help to maintain the mouthpiece 100a in a proper position relative to the mouth tray 500a, but may not maintain a secure attachment between the mouthpiece 100a and the mouth tray 500a. However, in use the user's mouth may wrap around the rear surface 106a of the mouthpiece 100a while the user's teeth are in the channels of the mouth tray 500a, and thus the user's mouth will maintain the mouthpiece 100a and the mouth tray 500a in an attached configuration during use.

In the exemplified embodiment, when the first and second bite protuberances 170a, 171a are located within the first and second recesses 570a, 571a, the top and bottom surfaces of the first and second bite protuberances 170a, 171a, may be flush with the upper and lower surfaces 521a, 522a of the bite platform 520a. This may ensure that a user will experience a seamless mouth feel despite the existence of the recesses 570a, 571a and the bite protuberances 170a, 171a nesting therein. However, the top and bottom surfaces of the first and second bite protuberances 170a, 171a may not be flush with the upper and lower surfaces 521a, 522a of the bite platform 520a in all embodiments, and other configurations are possible.

In alternative embodiments, other structures may be used to facilitate the attachment of the mouthpiece 100 to the mouth tray 500. For example, the mouthpiece 100 may comprise a clip feature located along at least a portion of the perimeter of the mouthpiece 100 that may clip onto the back of the mouth tray 500. For example, the clip feature may include an upper clip feature located along a top edge of the mouthpiece 100 for engagement with a top edge of the support wall 510 of the mouth tray 500 and a lower clip feature located along a lower edge of the mouthpiece 100 for engagement with a bottom edge of the support wall 510 of the mouth tray 500. In yet another embodiment, the mouth tray 500 may comprise a slot and the mouthpiece 100 of the whitening device 1000 may slide into the slot to facilitate the attachment.

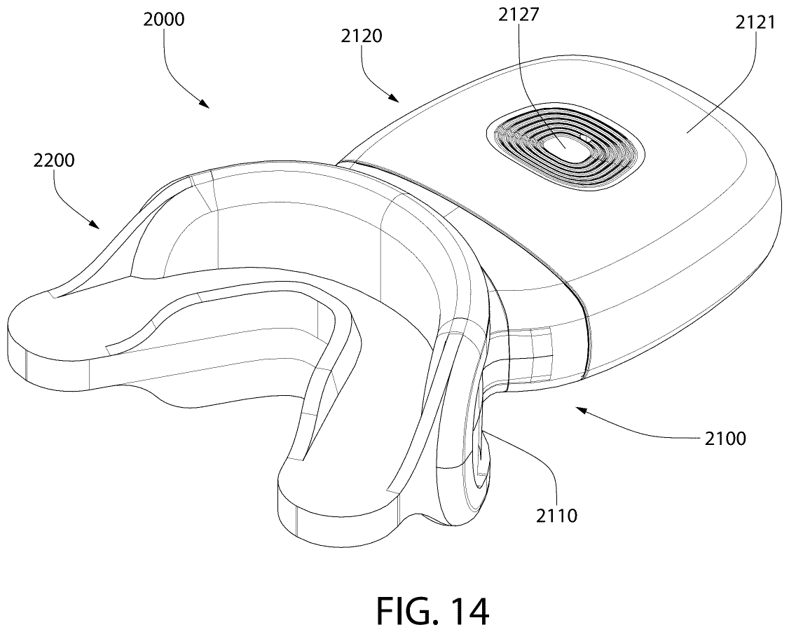

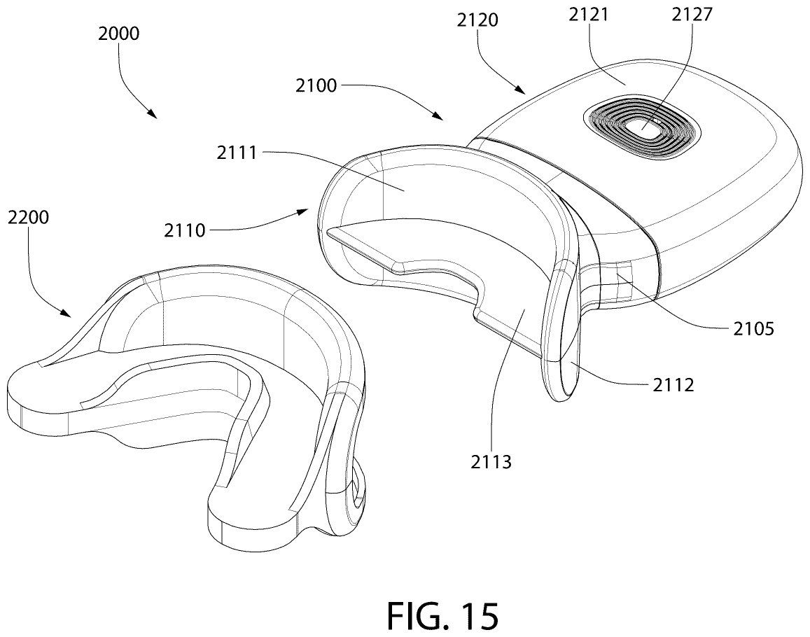

Referring to FIGS. 14 and 15, a tooth whitening system 2000 is illustrated in accordance with another embodiment of the present invention. The tooth whitening system 2000 generally comprises a whitening device 2100 and a mouth tray 2200 that are configured to be detachably coupled together. The main difference between this embodiment and the one previously described relates to the structure of the whitening device 2100. The whitening device 2100 is not a flimsy, flexible mouthpiece structure with a cord attached. Rather, the whitening device 2100 includes its own power source, such as a battery, such that the whitening device 2100 may be used without having to be plugged into an outlet. The whitening device 2100 may also include a power cord for charging the power source or for bypassing the power source to use external power in certain circumstances. However, the whitening device 2100 is capable of being used as a stand-alone device without being plugged into a wall outlet. Furthermore, while the whitening device 2100 is capable of being used without the mouth tray 2200, some users may prefer to use the whitening device 2100 with the mouth tray 2200 for one or more reasons. Thus, the mouth tray 2200 is an optional attachment to the whitening device 2100 that the end user can decide to use or not to use for a given treatment.

The whitening device 2100 is configured to emit electromagnetic radiation, such as light, onto the user's teeth. In some embodiments, the whitening device 2100 may be used to emit electromagnetic radiation onto a user's teeth in a pre-treatment step prior to applying a tooth whitening composition onto the user's teeth, and then again after the tooth whitening composition has been applied onto the user's teeth. In other embodiments, there may not be a pre-treatment step and the electromagnetic radiation may be applied onto the user's teeth with the tooth whitening device 2100 after the user's teeth are coated with a tooth whitening composition.

The whitening device 2100 may comprise a mouthpiece 2110 and a handle 2120. The mouthpiece 2110 may comprise a front surface 2111 through which the light is emitted and a rear surface 2112 opposite the front surface 2111. The front surface 2111 of the mouthpiece 110 may be concave. The rear surface 2112 of the mouthpiece 110 may be convex. The mouthpiece 2110 may comprise a bite platform 2113 protruding from the front surface 111. The bite platform 2113 may form a connection feature that is configured to facilitate an attachment of the whitening device 2100 to the mouth tray 2200.

The bite platform 2113 may be located centrally along a height of the front surface 2111 of the mouthpiece 2110 (measured from a bottom edge to a top edge of the front surface 2111) to divide the front surface 111 into an upper portion and a lower portion that are equal in size. The upper portion of the front surface 2111 may be in facing relation with the user's upper teeth and the lower portion of the front surface 2111 may be in facing relation with the user's lower teeth during use. The bite platform 2113 is depicted as a flat, plate-like structure in the exemplified embodiment. The bite platform 2113 may alternatively comprise multiple, spaced apart platforms protruding from the front surface 2111 of the whitening device 2100. The bite platform 2113 may comprise a distal wall extending from the distal end of the bite platform 2113 to help maintain the mouthpiece 2110 in the user's mouth during use (such a distal wall may be placed against the back or lingual surface of the tooth which faces the tongue). Thus, variations to the structure and shape of the bite platform 2113 are possible in accordance with the invention described herein. It should be noted that the term bite platform 2113 does not necessarily mean that the structure is always bitten by the user during use. The bite platform 2113 may be located between the user's teeth during some use scenarios, and in other use scenarios the bite platform 2113 may be located outside of the user's mouth and not between the user's teeth (such as when a cheek retractor assembly is used). For some users, the user may not feel that they are able to achieve a secure fit of the mouthpiece 2110 into their mouth while having only the bite platform 2113 to secure between the teeth. For those users, the mouth tray 2200 may be attached to the mouthpiece 2110 of the whitening device 2100 to improve the fit and feel for the user.

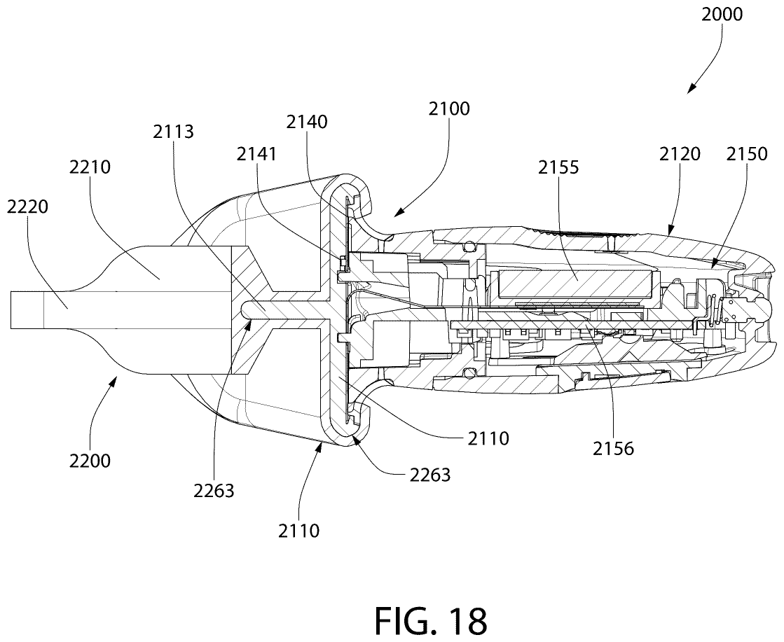

The whitening device 2100 further comprises the handle 2120 which protrudes or extends from the rear surface 2112 of the mouthpiece 2110. The handle 2120 may comprise a cavity within which various electronic components of the whitening device 2100 are located, as described below with reference to FIG. 18. The handle 2120 may comprise an outer surface 2121. The handle 2120 may comprise an actuator 2127. The actuator 2127 may be located on or along the top outer surface portion of the handle 2120. Alternatively, the actuator 2127 may be located along the bottom outer surface portion of the handle 2120. The actuator 2127 may be a depressible button that, when activated, activates light emitters of the whitening device 2100 to generate light and emit the light through the front surface 2111 of the mouthpiece 2110.

The whitening device 2100 may further comprise a transition portion 2105. The transition portion 2105 may be formed between the handle 2120 and the mouthpiece 2110, or the transition portion 2105 may be formed by the handle 2120 and the rear surface 2112 of the mouthpiece 2110 at the transition between the two. The transition portion 2105 may be an annular concave portion of the outer surface of the whitening device 2100 which may be formed by the shapes of the handle 2120 and the mouthpiece 2110 at the location where the two meet.

The handle 2120 may be a housing formed from a hard plastic material such as polypropylene, polyethylene, or similar. The mouthpiece 2110 may comprise an elastomeric outer layer to improve the feel of the mouthpiece 2110 in the mouth. Specifically, the mouthpiece 2110 may comprise a layer of an elastomeric material such as a thermoplastic elastomer or the like. In some embodiments, the mouthpiece 2110 may nonetheless be rigid rather than flexible, meaning that the arcuate shape of the mouthpiece 2110 may be fixed and not readily altered.

Referring briefly to FIG. 18, the whitening device 2100 will be further described. The whitening device 2100 may comprise a light emitting assembly 2140 that comprises one or more light emitters 2141. In the exemplified embodiment, the one or more light emitters 2141 comprises a plurality of light emitters 2141. In the exemplified embodiment, the plurality of light emitters 2141 are each discrete light emitting diodes or LEDs. The light emitters 2141 may be arranged in rows such that an upper row of the light emitters 2141 is configured to emit light onto the user's upper teeth while a lower row of the light emitters 2141 are configured to emit light onto the user's lower teeth. The light emitters 2141 in the rows may be aligned or offset to achieve a desired effect. The light emitters 2141 may be configured to generate light at a wavelength in a range between 380 nm and 440 nm, more specifically between 390 nm and 430 nm, still more specifically between 400 nm and 420 nm, and still more specifically between 405 nm and 415 nm.

The handle 2120 defines a cavity 2150 within which various electronic components may be located. Specifically, the whitening device 2100 may comprise a power source 2155 located in the cavity 2150. The power source 2155 may be operably coupled to the light emitters 2141 to power the light emitters 2155 when the whitening device 2100 is activated. In some embodiments, the power source 2155 may be a rechargeable battery that is rechargeable by a charging device that is coupled to a power outlet. In other embodiments, the power source 2155 may be a replaceable battery that is replaceable by an end user. Additional electronic components located in the cavity 2150 may include a circuit board 2156 to control operation of the whitening device 2100 when activated, such as parameters including on time, light intensity, wavelength, and the like.

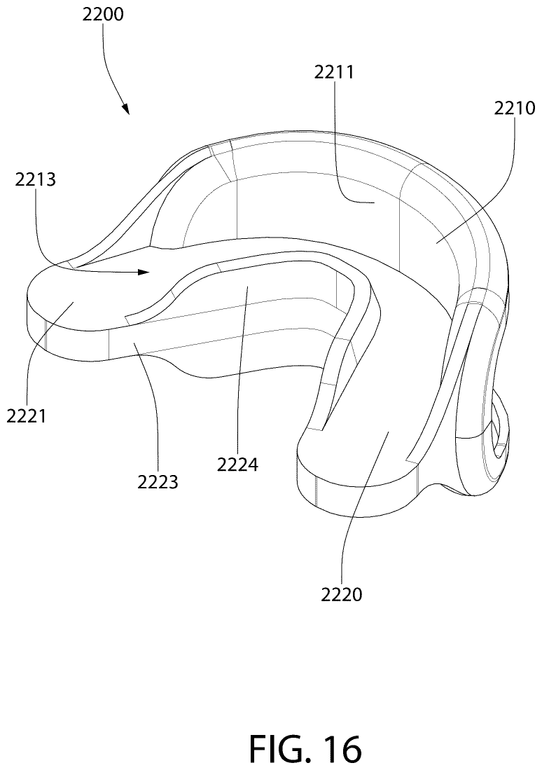

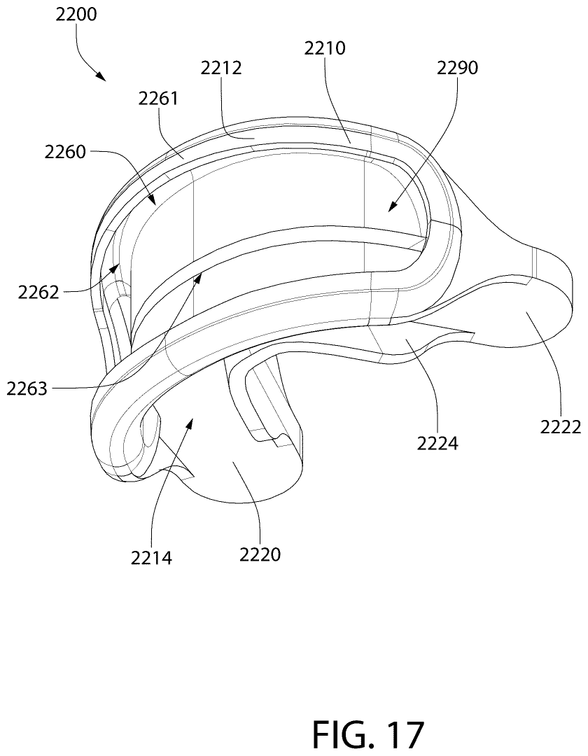

Referring to FIGS. 16 and 17, the mouth tray 2200 will be further described. The mouth tray 2200 is configured to be detachably coupled to the whitening device 2100 as described herein. Thus, the mouth tray 2200 may be configured to be inserted into the user's mouth during a whitening or other oral care treatment session. Thus, the mouth tray 2200 may be formed from a material that is safe to place into the user's mouth. The mouth tray 2200 may be formed from a hard plastic material, an elastomeric material, or the like in various different embodiments. The mouth tray 2200 may be formed from a transparent material in some embodiments. The mouth tray 2200 may be formed from a light transmissive material. The mouth tray 2200 may be formed from a material that allows light generated by the light emitters 2141 of the whitening device 2100 to pass through the mouth tray 2200 and onto the user's teeth during a treatment session.

The mouth tray 2200 may comprise a support wall 2210 having a front surface 2211 and a rear surface 2212, a bite platform 2220 extending from the front surface 2211 of the support wall 2210, and one or more connection features 2290 located on the rear surface 2212 of the support wall 2210. The front surface 2211 of the support wall 2210 may be concave and the rear surface 2212 of the support wall 2210 may be convex or planar. The support wall 2210 may be arcuate.

The bite platform 2220 may comprise an upper surface 2221, a lower surface 2222, and a distal end 2223. Furthermore, an end wall 2224 may protrude from the distal end 2223 of the bite platform 2220. The end wall 2224 may comprise an upper end wall portion that extends upwardly from the upper surface 2221 of the bite platform 2220 at or adjacent to the distal end 2223 and a lower end wall portion that extends downwardly from the lower surface 2222 of the bite platform 2220 at or adjacent to the distal and 2223. The end wall 2224 may extend along only a part of the bite platform 2220 as shown or the end wall 2224 may extend along an entirety of the distal end 2223. In an embodiment, the mouth tray 2200 comprises an upper teeth channel 2213 defined by the upper surface 2221 of the bite platform 2220 and an upper portion of the front surface 2211 of the support wall 2210. In an embodiment, the mouth tray 2200 comprises a lower teeth channel 2214 defined by the lower surface 2222 of the bite platform 2220 and a lower portion of the front surface 2211 of the support wall 2210. The upper teeth channel 2213 may be configured to receive the user's upper teeth during a treatment session. The lower teeth channel 2214 may be configured to receive the user's lower teeth during a treatment session.

The mouth tray 2200 may comprise a recess 2260 formed into the rear surface 2212 of the support wall 2210. An annular flange 2261 may extend over a periphery of the recess 2260 so that an annular pocket 2262 is formed along an outer peripheral portion of the recess 2260. Furthermore, an elongated slot 2263 may be formed into a floor of the recess 2260. The annular slot 2263, alone or in combination with the annular pocket 2262 and other parts of the recess 2260, may form the one or more connection features 2290. The elongated slot 2263 may be elongated in a horizontal direction. The elongated slot 2263 may extend through the support wall 2210 and into the bite platform 2220 in some embodiments.

Referring to FIG. 18, the attachment of the whitening device 2100 to the mouth tray 2200 will be described. The whitening device 2100 is attached to the mouth tray 2200 by inserting the bite platform 2113 of the whitening device 2100 into the elongated slot 2263 of the mouth tray 2200. A peripheral edge of the mouthpiece 2110 of the whitening device 2100 may nest within the annular pocket 2263 of the mouth tray 2200. The mouth tray 2200 may be formed from an elastomeric or otherwise flexible material to enable the mouth tray 2200 to fit over the mouthpiece 2110 as shown. The whitening device 2100 and the mouth tray 2200 may be attached together by a friction fit. Furthermore, the annular flange 2261 of the mouth tray 2200 may wrap around a portion of the mouthpiece 2110 of the whitening device 2100 to make the attachment between the two parts more secure. The mouth tray 2200 may be separated or detached from the whitening device 2100 by pulling the mouth tray 2200 and the whitening device 2100 apart. The bite platform 2113 of the whitening device 2100 may form a first connection feature and the elongated slot 2263 of the mouth tray 2200 may form a second connection feature that is configured to mate with the first engagement feature to facilitate the attachment of the whitening device 2100 to the mouth tray 2200.

As used throughout, ranges are used as shorthand for describing each and every value that is within the range. Any value within the range can be selected as the terminus of the range. In addition, all references cited herein are hereby incorporated by reference in their entireties. In the event of a conflict in a definition in the present disclosure and that of a cited reference, the present disclosure controls.

While the invention has been described with respect to specific examples including presently preferred modes of carrying out the invention, those skilled in the art will appreciate that there are numerous variations and permutations of the above described systems and techniques. It is to be understood that other embodiments may be utilized and structural and functional modifications may be made without departing from the scope of the present invention. Thus, the spirit and scope of the invention should be construed broadly as set forth in the appended claims.

### EXEMPLARY CLAIM SET

The following is a non-limiting list of exemplary claims:

Exemplary Claim 1. A tooth whitening system comprising: a whitening device comprising: a mouthpiece comprising a front surface and one or more first connection features;
and a light emitting assembly comprising one or more light emitters that are configured to generate light which is emitted from the front surface of the mouthpiece; a mouth tray comprising: a support wall comprising a front surface and a rear surface; a bite platform extending from the front surface of the support wall; and one or more second connection features; wherein the mouthpiece of the whitening device is configured to be detachably coupled to the mouth tray by engagement between the one or more first connection features of the mouthpiece and the one or more second connection features of the mouth tray.

Exemplary Claim 2. The tooth whitening system according to Exemplary Claim 1further comprising: the whitening device comprising a power cord extending from a rear surface of the mouthpiece that is opposite the front surface of the mouthpiece, the power cord comprising a plug for engaging a socket to power the one or more light emitters.

Exemplary Claim 3. The tooth whitening system according to Exemplary Claim 1 or Exemplary Claim 2 further comprising: the mouth tray comprising an upper teeth channel defined by an upper surface of the bite platform and an upper portion of the front surface of the support wall and a lower teeth channel defined by a lower surface of the bite platform and a lower portion of the front surface of the support wall, wherein the upper teeth channel is configured to receive a user's upper teeth and the lower teeth channel is configured to receive the user's lower teeth during use of the tooth whitening system.

Exemplary Claim 4. The tooth whitening system according to any one of Exemplary Claims 1 to 3 wherein the front surface of the mouthpiece is adjacent to or in contact with the rear surface of the support wall of the mouth tray when the mouthpiece is detachably coupled to the mouth tray, and wherein the support wall of the mouth tray is transparent so that light emitted from the front surface of the mouthpiece emits through the support wall of the mouth tray.

Exemplary Claim 5. The tooth whitening system according to any one of Exemplary Claims 1 to 4 wherein mouthpiece comprises a rear surface opposite the front surface, the front surface of the mouthpiece being concave and the rear surface of the mouthpiece being convex, and further wherein the rear surface of the support wall of the mouth tray being convex.

Exemplary Claim 6. The tooth whitening system according to any one of Exemplary Claims 1 to 5 wherein the mouthpiece comprises a front surface, a rear surface, a central portion, a first side portion located on a first side of the central portion, and a second side portion located on a second side of the central portion, the mouthpiece being flexible such that the first and second side portions are configured to bend or fold relative to the central portion about either of the front and rear surfaces of the mouthpiece.

Exemplary Claim 7. The tooth whitening system according to any one of Exemplary Claims 1 to 6 further comprising: the mouth tray being symmetrical about a central reference plane; the one or more second connection features of the mouth tray comprising a first capture element located on a first side of the central reference plane and a second capture element located on a second side of the central reference plane; the one or more first connection features of the mouthpiece comprising a pair of the first connection features, a first one of the pair of the first connection features being configured to be retained by the first capture element and the second one of the pair of the first connection features configured to be retained by the second capture element to detachably couple the mouthpiece to the mouth tray.

Exemplary Claim 8. The tooth whitening system according to Exemplary Claim 7 further comprising: the mouthpiece comprising a front surface, a rear surface, a central portion, a first side portion located on a first side of the central portion, and a second side portion located on a second side of the central portion, the first one of the pair of the first connection features comprising at least a portion of the first side portion of the mouthpiece, and the second one of the pair of the first connection features comprising at least a portion of the second side portion of the mouthpiece.

Exemplary Claim 9. The tooth whitening system according to Exemplary Claim 8 further comprising: the first capture element of the mouth tray comprising a first strap portion that extends over the rear surface of the first side portion of the mouthpiece thereby sandwiching the first side portion of the mouthpiece between the first strap portion of the first capture element of the mouth tray and the rear surface of the mouth tray; and the second capture element of the mouth tray comprising a second strap portion that extends over the rear surface of the second side portion of the mouthpiece thereby sandwiching the second side portion of the mouthpiece between the second strap portion of the second capture element of the mouth tray and the rear surface of the mouth tray.

Exemplary Claim 10. The tooth whitening system according to any one of Exemplary Claims 7 to 9 further comprising: the first capture element of the mouth tray comprising a first receptacle, the first one of the pair of the first connection features of the mouthpiece nesting within the first receptacle when the mouthpiece is coupled to the mouth tray; and the second capture element of the mouth tray comprising a second receptacle, the second one of the pair of the first connection features of the mouthpiece nesting within the second receptacle when the mouthpiece is coupled to the mouth tray.

Exemplary Claim 11. The tooth whitening system according to any one of Exemplary Claims 1 to 10 further comprising: the mouthpiece comprising at least one bite protuberance extending from the front surface of the mouthpiece, the one or more first connection features of the mouthpiece comprising the at least one bite protuberance; the mouth tray comprising at least one recess formed into the rear surface of the support wall; and wherein the at least one bite protuberance of the mouthpiece nests within the at least one recess of the mouth tray when the mouthpiece of the whitening device is detachably coupled to the mouth tray.

Exemplary Claim 12. The tooth whitening system according to Exemplary Claim 11 wherein the at least one recess formed into the rear surface of the support wall further extends into a rear portion of the bite platform of the mouth tray.

Exemplary Claim 13. The tooth whitening system according to Exemplary Claim 11 or Exemplary Claim 12 further comprising: the at least one bite protuberance comprising a first bite protuberance and a second bite protuberance that extend from the front surface of the mouthpiece in a spaced apart manner; the at least one recess of the mouth tray comprising a first recess and a second recess that are positioned in a spaced apart manner; and wherein when the mouthpiece of the whitening device is coupled to the mouth tray, the first bite protuberance nest within the first recess and the second bite protuberance nests within the second recess.

Exemplary Claim 14. The tooth whitening system according to any one of Exemplary Claims 1 to 13 wherein the one or more light emitters are configured to generate the light in a wavelength between 400 nm and 420 nm with a current greater than 6 mA.

Exemplary Claim 15. A tooth whitening kit comprising: a whitening device comprising: a mouthpiece comprising a front surface; a light emitting assembly comprising one or more light emitters that are configured to generate light which is emitted from the front surface of the mouthpiece; and one or more first connection features; a mouth tray comprising one or more second connection features, the mouth tray comprising an upper channel configured to receive a user's upper teeth during use and a lower channel configured to receive the user's lower teeth during use, wherein the whitening device and the mouth tray are configured to be detachably coupled together via engagement between the one or more first connection features of the whitening device and the one or more second connection features of the mouth tray; and at least one dispensing unit containing a tooth whitening composition.

Exemplary Claim 16. The tooth whitening kit according to Exemplary Claim 15 further comprising a package containing the whitening device, the mouth tray, and the at least one dispensing unit.

Exemplary Claim 17. The tooth whitening kit according to Exemplary Claim 15 or Exemplary Claim 16 further comprising: a sheet of material configured to be formed into a custom tray having one or more third connection features, wherein the whitening device is configured to be detachably coupled to the custom tray via engagement between the one or more first connection features of the whitening device and the one or more third connection features of the custom tray.

Exemplary Claim 18. The tooth whitening kit according to any one of Exemplary Claims 15 to 17 wherein the mouth tray is transparent so that light emitted from the front surface of the mouthpiece of the whitening device transmits through the mouth tray when the whitening device is detachably coupled to the mouth tray during use.

Exemplary Claim 19. The tooth whitening kit according to any one of Exemplary Claims 15 to 18 further comprising: the mouthpiece comprising a first side portion, a second side portion, and a central portion between the first and second side portions, the one or more first connection features of the whitening device comprising the first and second side portions of the mouthpiece; the mouth tray comprising a support wall having a front surface and a rear surface opposite the front surface, the one or more second connection features of the mouth tray comprising first and second strap elements located on the rear surface of the support wall of the mouth tray; and wherein the first and second side portions of the whitening device are folded relative to the central portion of the whitening device and then inserted into a space between the first and second strap elements, respectively, and the rear surface of the support wall of the mouth tray to facilitate the detachable coupling between the whitening device and the mouth tray.

Exemplary Claim 20. The tooth whitening kit according to any one of Exemplary Claims 15 to 18 further comprising: the mouthpiece comprising at least one bite protuberance extending from the front surface of the mouthpiece, the at least one first connection feature of the whitening device comprising the at least one bite protuberance; the mouth tray comprising a support wall having a front surface, a rear surface opposite the front surface, and a bite platform extending from the front surface, the one or more second connection features of the mouth tray comprising at least one recess extending from the rear surface of the support wall and into the bite platform; and wherein the whitening device is detachably coupled to the mouth tray by nesting the at least one bite protuberance of the mouthpiece within the at least one recess of the mouth tray.

Exemplary Claim 21. A tooth whitening method comprising: detachably coupling a whitening device to a mouth tray that is distinct from the whitening device, the whitening device comprising a mouthpiece comprising a front surface and at least one light emitter, the mouth tray comprising a support wall and a bite platform that define an upper channel and a lower channel; introducing a tooth whitening composition into the upper and lower channels of the mouth tray; inserting the mouth tray into a user's mouth so that upper teeth of the user are located within the upper channel of the mouth tray and lower teeth of the user are located within the lower channel of the mouth tray; and activating the whitening device so that the at least one light emitter generates light and emits the light from the front surface of the mouthpiece and through the support wall of the mouth tray and onto the upper and lower teeth of the user.

Exemplary Claim 22. The tooth whitening method according to Exemplary Claim 21 wherein the support wall of the mouth tray comprises a front surface and a rear surface, the bite platform protruding from the front surface of the support wall, and wherein the front surface of the mouthpiece of the whitening device is adjacent to the rear surface of the support wall when the whitening device is detachably coupled to the mouth tray.

Exemplary Claim 23. The tooth whitening method according to Exemplary Claim 21 or Exemplary Claim 22 further comprising: the mouthpiece comprising a first side portion, a second side portion, and a central portion located between the first and second side portions; the mouth tray comprising a first strap element and a second strap element located on a rear surface of the support wall; wherein the mouthpiece is detachably coupled to the mouth tray by: inserting the first side portion of the mouthpiece into a space between the first strap element and the rear surface of the support wall; and inserting the second side portion of the mouthpiece into a space between the second strap element and the rear surface of the support wall.

Exemplary Claim 24. The tooth whitening method according to Exemplary Claim 23 further comprising: folding the first side portion of the mouthpiece relative to the central portion of the mouthpiece prior to inserting the first side portion of the mouthpiece into the space between the first strap element and the rear surface of the support wall; and folding the second side portion of the mouthpiece relative to the central portion of the mouthpiece prior to inserting the second side portion of the mouthpiece into the space between the second strap element and the rear surface of the support wall.

Exemplary Claim 25. The tooth whitening method according to Exemplary Claim 21 or Exemplary Claim 22 further comprising: the mouthpiece comprising at least one bite protuberance protruding from the front surface; and the support wall of the mouth tray comprising a front surface and a rear surface opposite the front surface, the bite platform of the mouth tray extending from the front surface of the support wall, and further comprising at least one recess formed into the rear surface of the support wall of the mouth tray; and wherein the mouthpiece is detachably coupled to the mouth tray by inserting the at least one bite protuberance of the mouthpiece into the at least one recess of the support wall of the mouth tray.

Exemplary Claim 26. A tooth whitening system comprising: a whitening device comprising: a mouthpiece comprising a front surface; and a light emitting assembly comprising one or more light emitters that are configured to generate light which is emitted from the front surface of the mouthpiece; a mouth tray comprising: a support wall comprising a front surface and a rear surface; and a bite platform extending from the front surface of the support wall; wherein the whitening device is configured to be detachably coupled to the mouth tray by engagement between the mouthpiece of the whitening device and the support wall of the mouth tray.

Exemplary Claim 27. A tooth whitening system comprising: a whitening device comprising: a mouthpiece comprising a front surface; and a light emitting assembly comprising one or more light emitters that are configured to generate light which is emitted from the front surface of the mouthpiece; a mouth tray comprising: a support wall comprising a front surface and a rear surface; and a bite platform extending from the front surface of the support wall, the bite platform and the front surface of the support wall defining an upper channel and a lower channel; wherein in a first use configuration, the mouthpiece of the whitening device is positioned within a user's mouth and the one or more light emitters are powered to emit the light onto the user's teeth; and wherein in a second use configuration, the whitening device is coupled to the mouth tray, the mouth tray is positioned within the user's mouth so that upper teeth of the user are located within the upper channel of the mouth tray and lower teeth of the user are located within he lower channel of the mouth tray, and the one or more light emitters are powered to emit the light onto the user's teeth.

Exemplary Claim 28. The tooth whitening system according to Exemplary Claim 27 wherein the mouth tray is transparent and wherein the light generated from the one or more light emitters passes through the support wall of the mouth tray during the second use configuration.

Exemplary Claim 29. The tooth whitening system according to Exemplary Claim 27 or Exemplary Claim 28 wherein in the first use configuration, a tooth whitening composition is applied directly onto the user's teeth prior to positioning the mouthpiece of the whitening device within the user's mouth, and wherein in the second use configuration, the tooth whitening composition is introduced into the first channel and the second channel prior to positioning the mouth tray within the user's mouth.

Exemplary Claim 30. The tooth whitening system according to any one of Exemplary Claims 27 to 29 wherein in the second use configuration, the mouthpiece of the whitening device is coupled directly to the support wall of the mouth tray with the front surface of the mouthpiece facing the rear surface of the support wall.

What is claimed is:

1. A tooth whitening system comprising:
a whitening device comprising:
a mouthpiece comprising a front surface and one or more first connection features; and
a light emitting assembly comprising one or more light emitters that are configured to generate light which is emitted from the front surface of the mouthpiece;
a mouth tray comprising:
a support wall comprising a front surface and a rear surface;
a bite platform extending from the front surface of the support wall; and
one or more second connection features;
wherein the mouthpiece of the whitening device is configured to be detachably coupled to the mouth tray by engagement between the one or more first connection features of the mouthpiece and the one or more second connection features of the mouth tray.

2. The tooth whitening system according to claim 1 further comprising:
the whitening device comprising a power cord extending from a rear surface of the mouthpiece that is opposite the front surface of the mouthpiece, the power cord comprising a plug for engaging a socket to power the one or more light emitters.

3. The tooth whitening system according to claim 1 further comprising:
the mouth tray comprising an upper teeth channel defined by an upper surface of the bite platform and an upper portion of the front surface of the support wall and a lower teeth channel defined by a lower surface of the bite platform and a lower portion of the front surface of the support wall, wherein the upper teeth channel is configured to receive a user's upper teeth and the lower teeth channel is configured to receive the user's lower teeth during use of the tooth whitening system.

4. The tooth whitening system according to claim 1 wherein the front surface of the mouthpiece is adjacent to or in contact with the rear surface of the support wall of the mouth tray when the mouthpiece is detachably coupled to the mouth tray, and wherein the support wall of the mouth tray is transparent so that light emitted from the front surface of the mouthpiece emits through the support wall of the mouth tray.

5. The tooth whitening system according to claim 1 wherein mouthpiece comprises a rear surface opposite the front surface, the front surface of the mouthpiece being concave and the rear surface of the mouthpiece being convex, and further wherein the rear surface of the support wall of the mouth tray being convex.

6. The tooth whitening system according to claim 1 wherein the mouthpiece comprises a front surface, a rear surface, a central portion, a first side portion located on a first side of the central portion, and a second side portion located on a second side of the central portion, the mouthpiece being flexible such that the first and second side portions are configured to bend or fold relative to the central portion about either of the front and rear surfaces of the mouthpiece.

7. The tooth whitening system according to claim 1 further comprising:
the mouth tray being symmetrical about a central reference plane;
the one or more second connection features of the mouth tray comprising a first capture element located on a first side of the central reference plane and a second capture element located on a second side of the central reference plane;
the one or more first connection features of the mouthpiece comprising a pair of the first connection features, a first one of the pair of the first connection features being configured to be retained by the first capture element and the second one of the pair of the first connection features configured to be retained by the second capture element to detachably couple the mouthpiece to the mouth tray.

8. The tooth whitening system according to claim 7 further comprising:
the mouthpiece comprising a front surface, a rear surface, a central portion, a first side portion located on a first side of the central portion, and a second side portion located on a second side of the central portion, the first one of the pair of the first connection features comprising at least a portion of the first side portion of the mouthpiece, and the second one of the pair of the first connection features comprising at least a portion of the second side portion of the mouthpiece.

9. The tooth whitening system according to claim 8 further comprising:
the first capture element of the mouth tray comprising a first strap portion that extends over the rear surface of the first side portion of the mouthpiece thereby sandwiching the first side portion of the mouthpiece between the first strap portion of the first capture element of the mouth tray and the rear surface of the mouth tray; and
the second capture element of the mouth tray comprising a second strap portion that extends over the rear surface of the second side portion of the mouthpiece thereby sandwiching the second side portion of the mouthpiece between the second strap portion of the second capture element of the mouth tray and the rear surface of the mouth tray.

10. The tooth whitening system according to claim 7 further comprising:
the first capture element of the mouth tray comprising a first receptacle, the first one of the pair of the first connection features of the mouthpiece nesting within the first receptacle when the mouthpiece is coupled to the mouth tray; and
the second capture element of the mouth tray comprising a second receptacle, the second one of the pair of the first connection features of the mouthpiece nesting within the second receptacle when the mouthpiece is coupled to the mouth tray.

11. The tooth whitening system according to claim 1 further comprising:
the mouthpiece comprising at least one bite protuberance extending from the front surface of the mouthpiece, the one or more first connection features of the mouthpiece comprising the at least one bite protuberance;
the mouth tray comprising at least one recess formed into the rear surface of the support wall; and
wherein the at least one bite protuberance of the mouthpiece nests within the at least one recess of the mouth tray when the mouthpiece of the whitening device is detachably coupled to the mouth tray.

12. The tooth whitening system according to claim 1 wherein the one or more light emitters are configured to generate the light in a wavelength between 400 nm and 420 nm with a current greater than 6 mA.

13. A tooth whitening kit comprising:
a whitening device comprising:
a mouthpiece comprising a front surface;
a light emitting assembly comprising one or more light emitters that are configured to generate light which is emitted from the front surface of the mouthpiece; and
one or more first connection features;
a mouth tray comprising one or more second connection features, the mouth tray comprising an upper channel configured to receive a user's upper teeth during use and a lower channel configured to receive the user's lower teeth during use, wherein the whitening device and the mouth tray are configured to be detachably coupled together via engagement between the one or more first connection features of the whitening device and the one or more second connection features of the mouth tray; and
at least one dispensing unit containing a tooth whitening composition.

14. The tooth whitening kit according to claim 13 further comprising a package containing the whitening device, the mouth tray, and the at least one dispensing unit.

15. The tooth whitening kit according to claim 13 wherein the mouth tray is transparent so that light emitted from the front surface of the mouthpiece of the whitening device transmits through the mouth tray when the whitening device is detachably coupled to the mouth tray during use.

16. The tooth whitening kit according to claim 13 further comprising:
the mouthpiece comprising a first side portion, a second side portion, and a central portion between the first and second side portions, the one or more first connection features of the whitening device comprising the first and second side portions of the mouthpiece;
the mouth tray comprising a support wall having a front surface and a rear surface opposite the front surface, the one or more second connection features of the mouth tray comprising first and second strap elements located on the rear surface of the support wall of the mouth tray; and
wherein the first and second side portions of the whitening device are folded relative to the central portion of the whitening device and then inserted into a space between the first and second strap elements, respectively, and the rear surface of the support wall of the mouth tray to facilitate the detachable coupling between the whitening device and the mouth tray.

17. The tooth whitening kit according to any claim 13 further comprising:
the mouthpiece comprising at least one bite protuberance extending from the front surface of the mouthpiece, the at least one first connection feature of the whitening device comprising the at least one bite protuberance;
the mouth tray comprising a support wall having a front surface, a rear surface opposite the front surface, and a bite platform extending from the front surface, the one or more second connection features of the mouth tray comprising at least one recess extending from the rear surface of the support wall and into the bite platform; and
wherein the whitening device is detachably coupled to the mouth tray by nesting the at least one bite protuberance of the mouthpiece within the at least one recess of the mouth tray.

18. A tooth whitening system comprising:
a whitening device comprising:
a mouthpiece comprising a front surface; and
a light emitting assembly comprising one or more light emitters that are configured to generate light which is emitted from the front surface of the mouthpiece;
a mouth tray comprising:
a support wall comprising a front surface and a rear surface; and
a bite platform extending from the front surface of the support wall, the bite platform and the front surface of the support wall defining an upper channel and a lower channel;
wherein in a first use configuration, the mouthpiece of the whitening device is positioned within a user's mouth and the one or more light emitters are powered to emit the light onto the user's teeth; and
wherein in a second use configuration, the whitening device is coupled to the mouth tray, the mouth tray is positioned within the user's mouth so that upper teeth of the user are located within the upper channel of the mouth tray and lower teeth of the user are located within he lower channel of the mouth tray, and the one or more light emitters are powered to emit the light onto the user's teeth.

19. The tooth whitening system according to claim 18 wherein the mouth tray is transparent and wherein the light generated from the one or more light emitters passes through the support wall of the mouth tray during the second use configuration.

20. The tooth whitening system according to claim 18 wherein in the second use configuration, the mouthpiece of the whitening device is coupled directly to the support wall of the mouth tray with the front surface of the mouthpiece facing the rear surface of the support wall.
